(19) United States
(12) Patent Application Publication
(10) Pub. No.: US 2008/0242948 A1
(43) Pub. Date: Oct. 2, 2008

(54) Effective low-profile health monitoring or the like

(75) Inventors: Edward K.Y. Jung, Bellevue, WA (US); Eric C. Leuthardt, St. Louis, MO (US); Royce A. Levien, Lexington, MA (US); Robert W. Lord, Seattle, WA (US); Mark A. Malamud, Seattle, WA (US)

Correspondence Address: SEARETE LLC;CLARENCE T. TEGREENE, 1756 - 114TH AVE., S.E., SUITE 110, BELLEVUE, WA, 98004, US

(73) Assignee: Searete LLC, a limited liability corporation of the State of Delaware

(21) Appl. No.: 11/731,801

(22) Filed: Mar. 30, 2007

(51) Int. Cl. A61B 5/00

(52) U.S. Cl. 600/300

(57) ABSTRACT

A system, method, computer program product, and carrier are described for obtaining data from occupational or leisure intercommunication at least between a device and a user or receiving health-status-indicative data surreptitiously captured from an interaction between a device and a user; and applying one or more data extraction criteria to the health-status-indicative data surreptitiously captured from the interaction between the device and the user or signaling a data distillation indicating a health status change of the user at least partly based on the data from the occupational or leisure intercommunication.

### CROSS-REFERENCE TO RELATED APPLICATIONS

The present application is related to and claims the benefit of the earliest available effective filing date(s) from the following listed application(s) (the “Related Applications”) (e.g., claims earliest available priority dates for other than provisional patent applications or claims benefits under 35 USC § 119(e) for provisional patent applications, for any and all parent, grandparent, great-grandparent, etc. applications of the Related Application(s)).

### RELATED APPLICATIONS

For purposes of the USPTO extra-statutory requirements, the present application constitutes a continuation-in-part of U.S. patent application Ser. No. [To Be Assigned], entitled CONFIGURING SOFTWARE FOR EFFECTIVE HEALTH MONITORING OR THE LIKE, naming Edward K. Y. Jung, Eric C. Leuthardt; Royce A. Levien, Robert W. Lord and Mark A. Malamud as inventors, filed 30 Mar. 2007, which is currently co-pending, or is an application of which a currently co-pending application is entitled to the benefit of the filing date [Attorney Docket No. 0406-002-002-000000].

For purposes of the USPTO extra-statutory requirements, the present application constitutes a continuation-in-part of U.S. patent application Ser. No. [To Be Assigned], entitled EFFECTIVE RESPONSE PROTOCOLS FOR HEALTH MONITORING OR THE LIKE, naming Edward K. Y. Jung, Eric C. Leuthardt; Royce A. Levien, Robert W. Lord and Mark A. Malamud as inventors, filed 30 Mar. 2007, which is currently co-pending, or is an application of which a currently co-pending application is entitled to the benefit of the filing date [Attorney Docket No. 0406-002-003-000000].

The United States Patent Office (USPTO) has published a notice to the effect that the USPTO's computer programs require that patent applicants reference both a serial number and indicate whether an application is a continuation or continuation-in-part. Stephen G. Kunin, Benefit of Prior-Filed Application, USPTO Official Gazette Mar. 18, 2003, available at http://www.uspto.gov/web/offices/com/sol/og/2003/week 11/patbene.htm. The present Applicant Entity (hereinafter “Applicant”) has provided above a specific reference to the application(s) from which priority is being claimed as recited by statute. Applicant understands that the statute is unambiguous in its specific reference language and does not require either a serial number or any characterization, such as “continuation” or “continuation-in-part,” for claiming priority to U.S. patent applications. Notwithstanding the foregoing, Applicant understands that the USPTO's computer programs have certain data entry requirements, and hence Applicant is designating the present application as a continuation-in-part of its parent applications as set forth above, but expressly points out that such designations are not to be construed in any way as any type of commentary and/or admission as to whether or not the present application contains any new matter in addition to the matter of its parent application(s).

All subject matter of the Related Applications and of any and all parent, grandparent, great-grandparent, etc. applications of the Related Applications is incorporated herein by reference to the extent such subject matter is not inconsistent herewith.

### SUMMARY

In one aspect, a method includes but is not limited to obtaining data from occupational or leisure intercommunication at least between a device and a user and signaling a data distillation indicating a health status change of the user at least partly based on the data from the occupational or leisure intercommunication. In addition to the foregoing, other method aspects are described in the claims, drawings, and text forming a part of the present disclosure.

In one or more various aspects, related systems include but are not limited to circuitry and/or programming for effecting the herein-referenced method aspects; the circuitry and/or programming can be virtually any combination of hardware, software, and/or firmware configured to effect the herein-referenced method aspects depending upon the design choices of the system designer.

In one aspect, a system includes but is not limited to circuitry for obtaining data from occupational or leisure intercommunication at least between a device and a user and circuitry for signaling a data distillation indicating a health status change of the user at least partly based on the data from the occupational or leisure intercommunication. In addition to the foregoing, other system aspects are described in the claims, drawings, and text forming a part of the present disclosure.

In one aspect, a method includes but is not limited to receiving health-status-indicative data surreptitiously captured from an interaction between a device and a user and applying one or more data extraction criteria to the health-status-indicative data surreptitiously captured from the interaction between the device and the user. In addition to the foregoing, other method aspects are described in the claims, drawings, and text forming a part of the present disclosure.

In one or more various aspects, related systems include but are not limited to circuitry and/or programming for effecting the herein-referenced method aspects; the circuitry and/or programming can be virtually any combination of hardware, software, and/or firmware configured to effect the herein-referenced method aspects depending upon the design choices of the system designer.

In one aspect, a system includes but is not limited to circuitry for receiving health-status-indicative data surreptitiously captured from an interaction between a device and a user and circuitry for applying one or more data extraction criteria to the health-status-indicative data surreptitiously captured from the interaction between the device and the user. In addition to the foregoing, other system aspects are described in the claims, drawings, and text forming a part of the present disclosure.

In one aspect, a method includes but is not limited to obtaining an indication of an activity status change of an application program and causing a movement of data distillation code relating to physiology-indicative data in response to the indication of the activity status change of the application program. In addition to the foregoing, other method aspects are described in the claims, drawings, and text forming a part of the present disclosure.

In one or more various aspects, related systems include but are not limited to circuitry and/or programming for effecting the herein-referenced method aspects; the circuitry and/or programming can be virtually any combination of hardware, software, and/or firmware configured to effect the herein-referenced method aspects depending upon the design choices of the system designer.

In one aspect, a system includes but is not limited to circuitry for obtaining an indication of an activity status change of an application program and circuitry for causing a movement of data distillation code relating to physiology-indicative data in response to the indication of the activity status change of the application program. In addition to the foregoing, other system aspects are described in the claims, drawings, and text forming a part of the present disclosure.

In one aspect, a method includes but is not limited to obtaining (a) first clinical data acceptable to a clinical analysis module and (b) a pointer to the clinical analysis module, configuring one or more applications using the pointer to the clinical analysis module after receiving at least the first clinical data acceptable to the clinical analysis module, and using second clinical data acceptable to the clinical analysis module with at least one of the one or more applications configured using the pointer to the clinical analysis module after receiving at least the first clinical data acceptable to the clinical analysis module. In addition to the foregoing, other method aspects are described in the claims, drawings, and text forming a part of the present disclosure.

In one or more various aspects, related systems include but are not limited to circuitry and/or programming for effecting the herein-referenced method aspects; the circuitry and/or programming can be virtually any combination of hardware, software, and/or firmware configured to effect the herein-referenced method aspects depending upon the design choices of the system designer.

In one aspect, a system includes but is not limited to circuitry for obtaining (a) first clinical data acceptable to a clinical analysis module and (b) a pointer to the clinical analysis module, circuitry for configuring one or more applications using the pointer to the clinical analysis module after receiving at least the first clinical data acceptable to the clinical analysis module, and circuitry for using second clinical data acceptable to the clinical analysis module with at least one of the one or more applications configured using the pointer to the clinical analysis module after receiving at least the first clinical data acceptable to the clinical analysis module. In addition to the foregoing, other system aspects are described in the claims, drawings, and text forming a part of the present disclosure.

In one aspect, a method includes but is not limited to receiving an indication of an anomalous device-interactive performance of a specific individual and selecting one or more diagnostic instructions at least partly based on the anomalous device-interactive performance of the specific individual. In addition to the foregoing, other method aspects are described in the claims, drawings, and text forming a part of the present disclosure.

In one or more various aspects, related systems include but are not limited to circuitry and/or programming for effecting the herein-referenced method aspects; the circuitry and/or programming can be virtually any combination of hardware, software, and/or firmware configured to effect the herein-referenced method aspects depending upon the design choices of the system designer.

In one aspect, a system includes but is not limited to circuitry for receiving an indication of an anomalous device-interactive performance of a specific individual and circuitry for selecting one or more diagnostic instructions at least partly based on the anomalous device-interactive performance of the specific individual. In addition to the foregoing, other system aspects are described in the claims, drawings, and text forming a part of the present disclosure.

In one aspect, a method includes but is not limited to signaling a decision whether to notify a first party at least by applying a first screen to one or more health-status-indicative updates relating to a second party and signaling a decision whether to notify a third party at least by applying a second screen to the one or more health-status-indicative updates relating to the second party. In addition to the foregoing, other method aspects are described in the claims, drawings, and text forming a part of the present disclosure.

In one or more various aspects, related systems include but are not limited to circuitry and/or programming for effecting the herein-referenced method aspects; the circuitry and/or programming can be virtually any combination of hardware, software, and/or firmware configured to effect the herein-referenced method aspects depending upon the design choices of the system designer.

In one aspect, a system includes but is not limited to circuitry for signaling a decision whether to notify a first party at least by applying a first screen to one or more health-status-indicative updates relating to a second party and circuitry for signaling a decision whether to notify a third party at least by applying a second screen to the one or more health-status-indicative updates relating to the second party. In addition to the foregoing, other system aspects are described in the claims, drawings, and text forming a part of the present disclosure.

In addition to the foregoing, various other method and/or system and/or program product and/or physical carrier aspects are set forth and described in the teachings such as text (e.g., claims and/or detailed description) and/or drawings of the present disclosure.

The foregoing is a summary and thus contains, by necessity, simplifications, generalizations and omissions of detail; consequently, those skilled in the art will appreciate that the summary is illustrative only and is NOT intended to be in any way limiting. Other aspects, features, and advantages of the devices and/or processes and/or other subject matter described herein will become apparent in the teachings set forth herein.

### BRIEF DESCRIPTION OF THE FIGURES

FIG. 1 depicts an exemplary environment in which one or more technologies may be implemented.

FIG. 2 depicts a high-level logic flow of an operational process.

FIG. 3 depicts an exemplary environment in which one or more technologies may be implemented.

FIG. 4 depicts a high-level logic flow of an operational process.

FIG. 5 depicts an exemplary environment in which one or more technologies may be implemented.

FIG. 6 depicts a high-level logic flow of an operational process.

FIG. 7 depicts an exemplary environment in which one or more technologies may be implemented.

FIG. 8 depicts a high-level logic flow of an operational process.

FIG. 9 depicts an exemplary environment in which one or more technologies may be implemented.

FIG. 10 depicts a high-level logic flow of an operational process.

FIG. 11 depicts an exemplary environment in which one or more technologies may be implemented.

FIG. 12 depicts a high-level logic flow of an operational process.

FIGS. 13-20 depict exemplary environments in which one or more technologies may be implemented.

FIGS. 21-23 depict variants of the flow of FIG. 2.

FIGS. 24-25 depict variants of the flow of FIG. 6.

FIG. 26 depicts variants of the flow of FIG. 8.

FIGS. 27-28 depict variants of the flow of FIG. 10.

FIG. 29 depicts variants of the flow of FIG. 12.

FIG. 30 depicts variants of the flow of FIG. 4.

### DETAILED DESCRIPTION

Those having skill in the art will recognize that the state of the art has progressed to the point where there is little distinction left between hardware and software implementations of aspects of systems; the use of hardware or software is generally (but not always, in that in certain contexts the choice between hardware and software can become significant) a design choice representing cost vs. efficiency tradeoffs. Those having skill in the art will appreciate that there are various vehicles by which processes and/or systems and/or other technologies described herein can be effected (e.g., hardware, software, and/or firmware), and that the preferred vehicle will vary with the context in which the processes and/or systems and/or other technologies are deployed. For example, if an implementer determines that speed and accuracy are paramount, the implementer may opt for a mainly hardware and/or firmware vehicle; alternatively, if flexibility is paramount, the implementer may opt for a mainly software implementation; or, yet again alternatively, the implementer may opt for some combination of hardware, software, and/or firmware. Hence, there are several possible vehicles by which the processes and/or devices and/or other technologies described herein may be effected, none of which is inherently superior to the other in that any vehicle to be utilized is a choice dependent upon the context in which the vehicle will be deployed and the specific concerns (e.g., speed, flexibility, or predictability) of the implementer, any of which may vary. Those skilled in the art will recognize that optical aspects of implementations will typically employ optically-oriented hardware, software, and or firmware.

In the following detailed description, reference is made to the accompanying drawings, which form a part hereof. The use of the same symbols in different drawings typically indicates similar or identical items. The illustrative embodiments described in the detailed description, drawings, and claims are not meant to be limiting. Other embodiments may be utilized, and other changes may be made, without departing from the spirit or scope of the subject matter presented here.

Following are a series of systems and flowcharts depicting implementations of processes. For ease of understanding, the flowcharts are organized such that the initial flowcharts present implementations via an initial “big picture” viewpoint and thereafter the following flowcharts present alternate implementations and/or expansions of the “big picture” flowcharts as either sub-steps or additional steps building on one or more earlier-presented flowcharts. Those having skill in the art will appreciate that the style of presentation utilized herein (e.g., beginning with a presentation of a flowchart(s) presenting an overall view and thereafter providing additions to and/or further details in subsequent flowcharts) generally allows for a rapid and easy understanding of the various process implementations. In addition, those skilled in the art will further appreciate that the style of presentation used herein also lends itself well to modular and/or object-oriented program design paradigms.

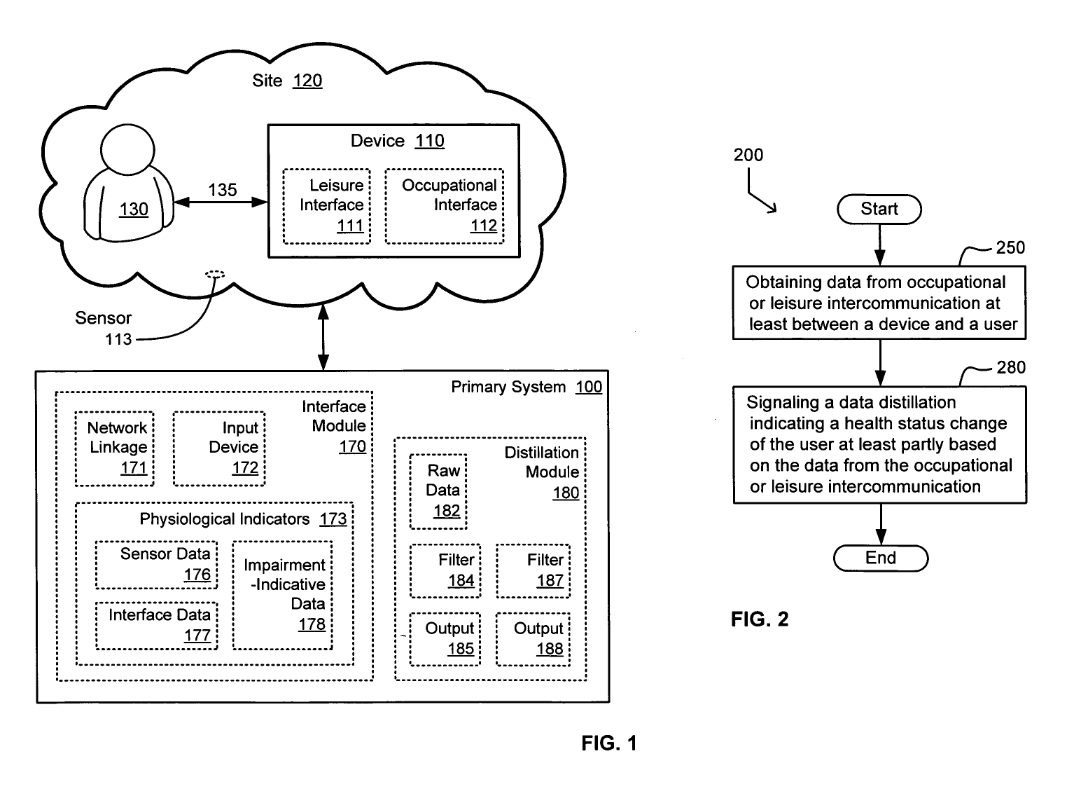

With reference now to FIG. 1, shown is an example of a system that may serve as a context for introducing one or more processes and/or devices described herein. As shown primary system 100 can communicate with site 120 at which user 130 can have (or have had) intercommunication 135 with device 110. Device 110 can include one or more of leisure interface 111 or occupational interface 112 to facilitate at least some occupational or leisure intercommunication detectable to one or more instances of sensor 113.

As shown, primary system 100 can include one or more of interface module 170 or distillation module 180. Interface module 170 can include one or more instances of network linkage 171, input device 172, or physiological indicators 173. Such indicators can, for example, include one or more instances of sensor data 176 (from sensor 113, e.g.), interface data 177, or impairment-indicative data 178. Distillation module 180 can include one or more instances of raw data 182, filters 184, 187, or outputs 185, 188.

With reference now to FIG. 2, there is shown a high-level logic flow 200 of an operational process. Flow 200 includes operation 250—obtaining data from occupational or leisure intercommunication at least between a device and a user (e.g. interface module 170 receiving sensor data 176 from sensor 113 or interface data 177 via device 110 at least from intercommunication 135). In some embodiments, sensor data 176 or interface data 177 thus obtained can also indicate other kinds of communications (with other parties or devices, e.g.) or other events. Alternatively or additionally, some or all of the data can be obtained in the form of raw data 182 as exemplified herein.

Flow 200 further includes operation 280—signaling a data distillation indicating a health status change of the user at least partly based on the data from the occupational or leisure intercommunication (e.g. distillation module 180 indicating substantial changes in impairment-indicative data 178 or other physiological indicators 173 relating to user 130). In some embodiments, one or more instances of sensors 113 obtain data from site 120 while stationary, observing user 130 (and perhaps other visitors to site 120). In some variants, one or more filters 184, 187 respectively provide one or more distillation outputs 185, 188 that may reasonably be expected to evaluate, consolidate, organize, or otherwise facilitate an interpretation of at least some of raw data 182 or other data. In a variant in which device 110 or sensor 113 includes a chemical or other biometric sensor operable to detect alcohol-containing breath, pale or clammy skin, a fast heartbeat or other difference relative to an earlier or later measurement, such changes can indicate a health status change susceptible of recordation and even automatic detection as described herein. Such signaling can be directed to one or more of user 130, caregivers or third parties, an archiving system or other communication system, or the like.

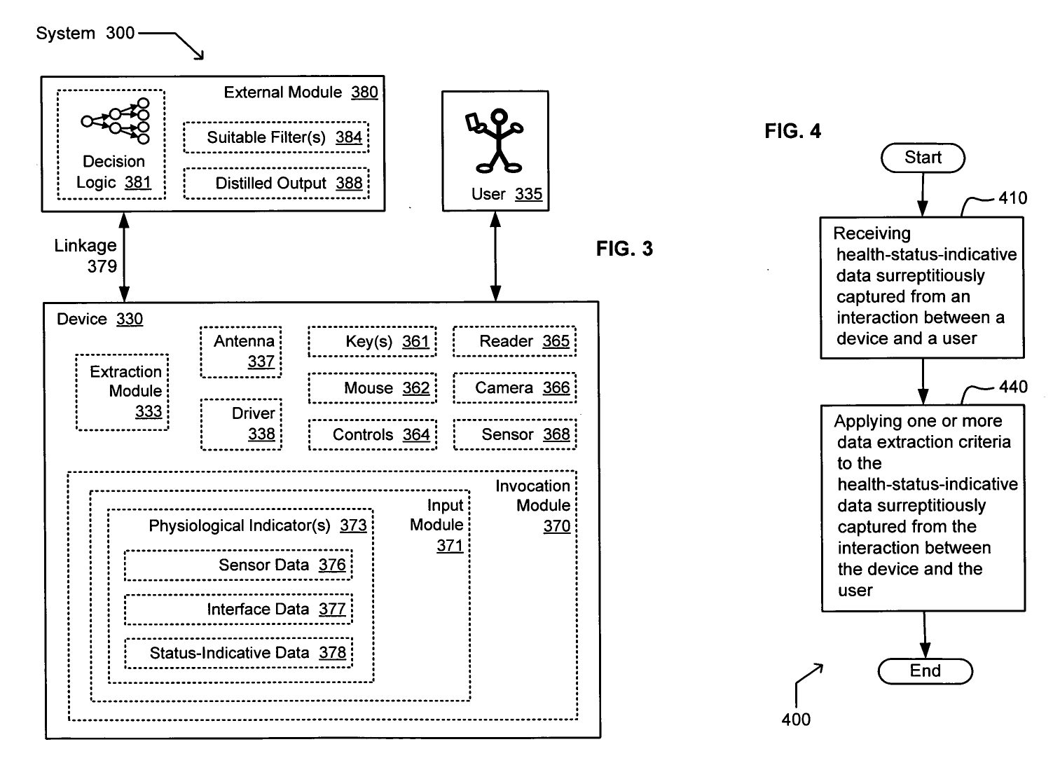

With reference now to FIG. 3, shown is an example of a system that may serve as a context for introducing one or more processes and/or devices described herein. As shown system 300 includes device 330 able to interact with user 335 or with external module 380. External module 380 can include one or more instances of decision logic 381, suitable filters 384, or distilled output 388. In communicating with external module 380, device 330 can optionally be configured to receive data through linkage 379 (via antenna 337, e.g.) or send data through linkage 379 (via driver 338, e.g.). Linkage 379 can include one or more conduits, wireless signal paths, or the like. Device 330 can likewise (optionally) include one or more instances of extraction module 333, keys 361, mouse 362, controls 364, readers 365, cameras 366, sensors 368, or invocation modules 370. Invocation module 370 can include input module(s) 371 with one or more physiological indicators 373 in the form of sensor data 376, interface data 377, status-indicative data 378, or the like.

With reference now to FIG. 4, there is shown a high-level logic flow 400 of an operational process. Flow 400 includes operation 410—receiving health-status-indicative data surreptitiously captured from an interaction between a device and a user (e.g. input module 371 or the like gathering data from a device interaction with user 335 engaged in everyday tasks or otherwise not consciously aware of the data being gathered). The health-status-indicative data can contain one or more physiological indicators 373, for example, comprising sensor data 376, interface data 377, or other status-indicative data 378 that may relate to user 335.

In some embodiments, such data is captured “surreptitiously” by virtue of the device participating in the interaction without a conspicuous notice of a capture event. (The user can authorize such surreptitious data captures before or after the capture events, for example.) Such modes of data acquisition can avoid error arising from consciousness of observation, for example, especially for situations in which a full-blown clinical intake concerning the interaction would be a burden. In some circumstances the surreptitiously captured data can be received from a pre-existing source such as a message archive, video data repository, or the like.

In the environment of FIG. 3, for example, the data can be surreptitiously captured by user 335 using one or more of key(s) 361, a mouse 362 or other pointing device, other controls 364; or via observations by one or more instances of readers 365 (such as by optical character recognition, e.g.), cameras 366, other sensors 368; or the like. In the environment of FIG. 1, alternatively or additionally, sensor 113 or device 110 with which a user interacts can optionally provide such data surreptitiously.

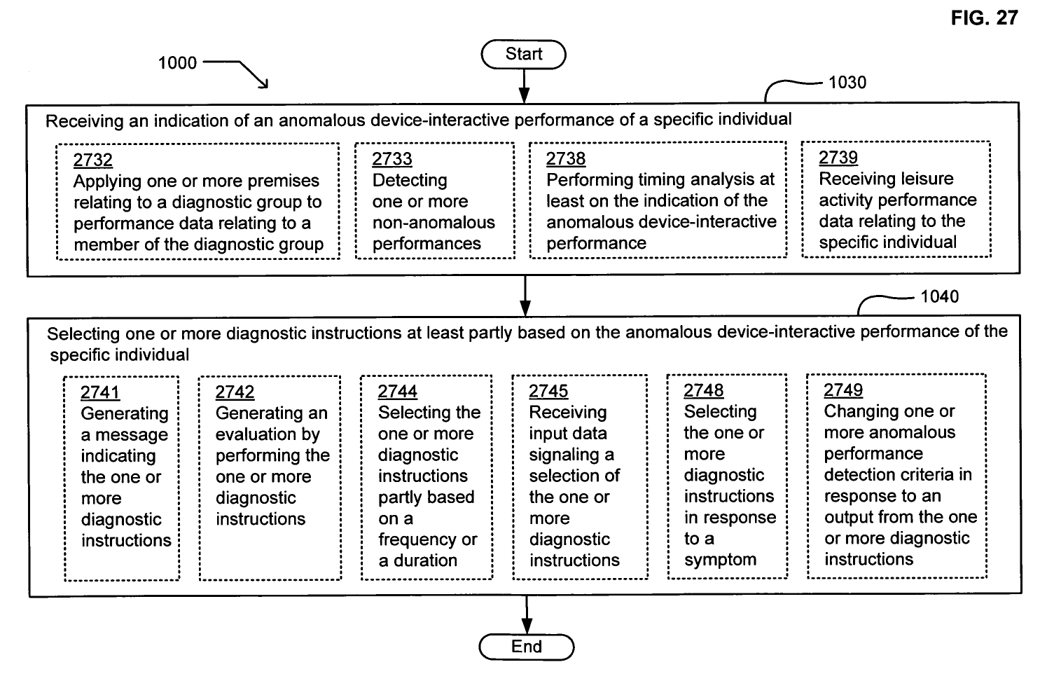

Flow 400 further includes operation 440—applying one or more data extraction criteria to the health-status-indicative data surreptitiously captured from the interaction between the device and the user (e.g. invocation module 370 and one or more instances of decision logic 381, suitable filters 384, or other extraction modules 333 generating distilled output 388 as described herein). In some embodiments, decision logic 381 can optionally be configured to select diagnostic instructions, for example, based on any anomalous data from the surreptitiously captured data according to flows in FIGS. 10, 27, or 28 as described in detail below. Distilled output 388 can thereby or thereafter be summarized, recorded, analyzed, or the like in various modes as described below.

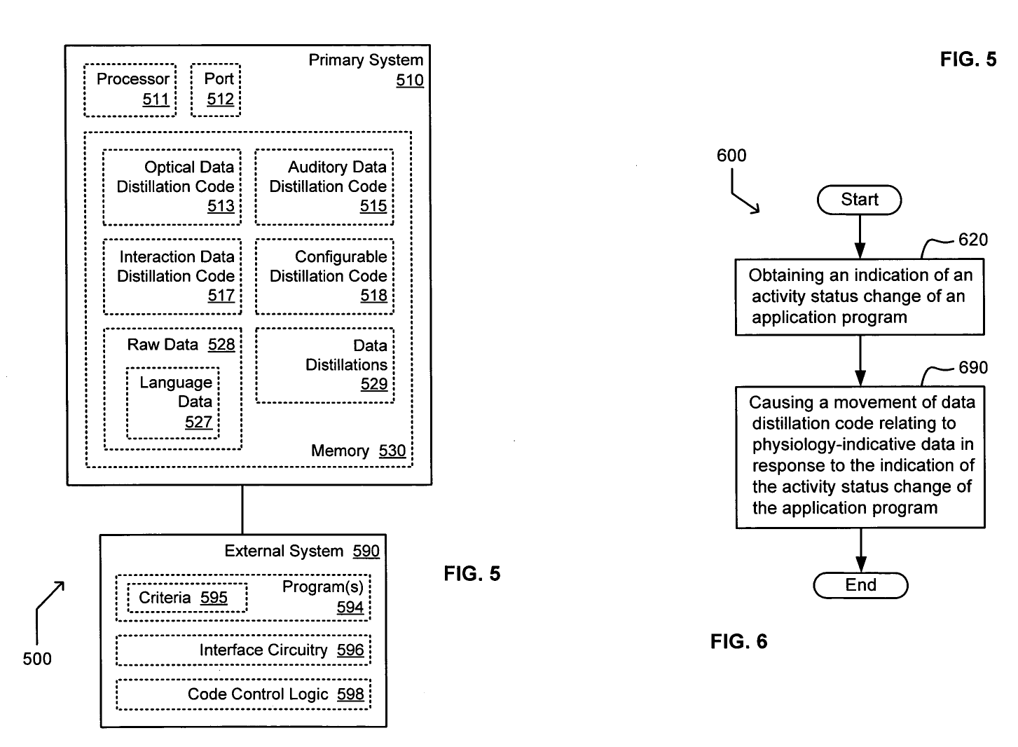

With reference now to FIG. 5, shown is an example of a system that may serve as a context for introducing one or more processes and/or devices described herein. As shown system 500 can include one or more instances of primary system 510 and external system 590 operably coupled. Primary system 510 can include one or more instances of processors 511, ports 512, or memory 530. Memory 530 can include (in respective “memories,” e.g.) one or more instances of optical data distillation code 513, auditory data distillation code 515, interaction data distillation code 517, configurable distillation code 518, language data 527 or other raw data 528, or data distillations 529. External system 590 can include one or more instances of programs 594 with criteria 595, interface circuitry 596, or code control logic 598.

With reference now to FIG. 6, there is shown a high-level logic flow 600 of an operational process. Flow 600 includes operation 620—obtaining an indication of an activity status change of an application program (e.g. port 512 receiving or otherwise detecting an indication that one or more user programs 594 have come online, stalled, encountered an error, completed a task, or the like). If a user powers up external system 590, for example, this can cause one or more programs 594 to be loaded or otherwise trigger such activity status changes in some configurations.

In some embodiments, “physiology-indicative” data includes any information that a knowledgeable caregiver, patient, expert system or other diagnostic agent might reasonably recognize as relevant for monitoring or understanding some attribute of test subjects or their circumstances. As exemplified herein, the physiology-indicative data can include one or more of a performance record, a measurement, or other information of potential relevance to analyzing physiological attributes of individuals such as device users, patients, workers, employers, or the like who observe or provide information to application programs. The physiology-indicative data can optionally include voice or other auditory data, pupil dilation or other optical data, device movements or other user-entered data, position information (of body parts, e.g.), or chemical or electrical sensor signals or the like to indicate data from one or more individuals. Alternatively or additionally, the physiology-indicative data can include a date or time, screen display expressions, code configuration attributes, hardware attributes, resource attributes or other information from devices and systems.

Flow 600 further includes operation 690—causing a movement of data distillation code relating to physiology-indicative data in response to the indication of the activity status change of the application program (e.g. processor 511 or code control logic 598 causing a movement of auditory data distillation code 515 into memory 530 so that it can process one or more data distillations 529 from language data 527 or other raw data 528). In some embodiments, auditory data distillation code 515 can generate one or more data distillations 529 by removing, marking, or otherwise deemphasizing portions of an audio recording below a given decibel threshold, for example, such as a threshold of human hearing or lower. Auditory data distillation code 515 can, alternatively or additionally, present or otherwise highlight more-relevant or other anomalous auditory data such as coughing, snoring or wheezing (at least partly indicative of a respiratory disorder, e.g.); or frequent shouting, cursing, or crying (at least partly indicative of a physiological or other emotional disorder, e.g.). In some embodiments, auditory data distillation code 515 can likewise detect the device user(s) showing an increased success in responding to very soft sounds (indicative of a hearing improvement such as may result from a hearing aid or other treatment, e.g.) or exercising an increased vocabulary or performance in a memory game (at least slightly indicative of a cognitive improvement, e.g.); or the like. Those skilled in the art will recognize that speech and other patterns such as these can be recognized using commonly available algorithms, permitting implementation of a diverse array of variants of flow 600 in light of these teachings, without undue experimentation. In some embodiments, moreover, data distillations 529 can include a raw data sample, a link or other data indexing feature, or other logic for facilitating access to a portion of raw auditory data having a higher-than-nominal apparent relevance in relation to such symptoms and other indications of physiological relevance.

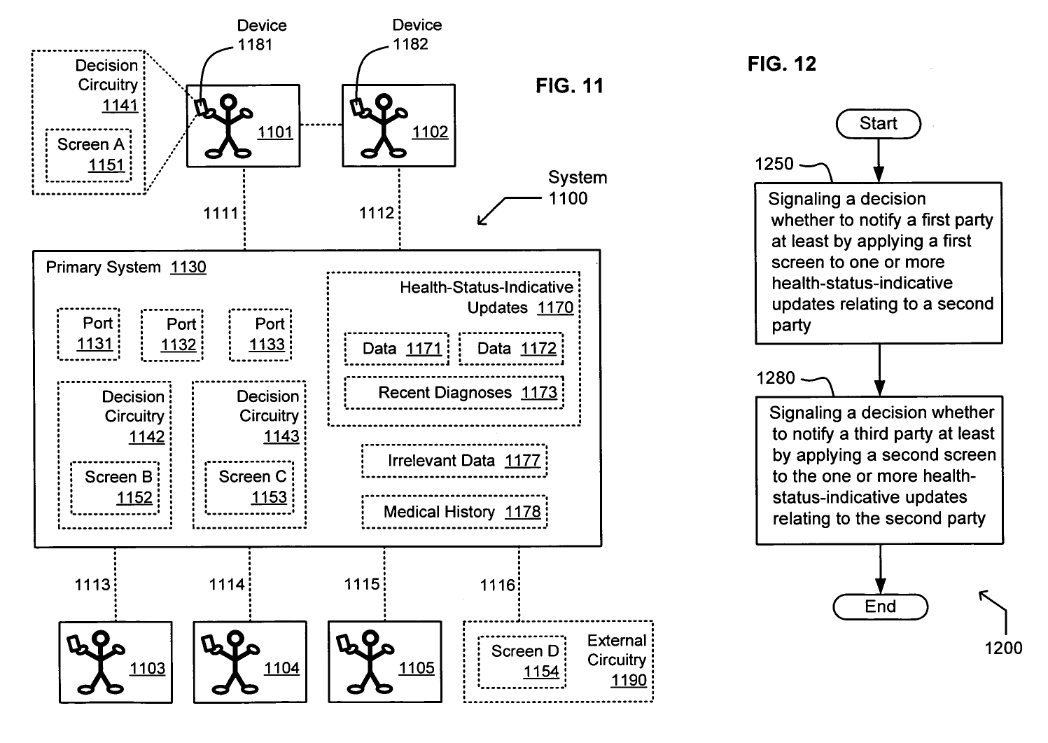

Alternatively or additionally, processor 511 can be configured to move one or more instances of optical data distillation code 513, interaction data distillation code 517, configurable distillation code 518 or the like into or out of memory 530 in response to the indication. Such data distillation code can (optionally) generate an evaluation or summary of some physiologically relevant data, for example, or otherwise at least partly filter out a less-relevant portion of such data or preferentially include more-relevant portions of such data. See, for example, the detailed explanations below in relation to FIG. 11.

In some embodiments, the data distillation code can include a segment containing at least one instruction operable for generating, selectively retaining, or more readily accessing a portion of the data with a more-than-nominal apparent utility, relative to another portion of the data. A block of data with an apparently decreasing relevance, for example, can (optionally) be distilled by displaying or ranking a first portion first, by removing or de-emphasizing a later portion, or by sampling, highlighting, or indexing early portions at a higher sampling frequency. In some embodiments, normal or otherwise duplicative data is extracted or a diverse sampling of data is otherwise preferentially retained. Some embodiments may likewise distill frequency indicators or other evaluations signifying prevalence of an observation to indicate a higher or lower apparent utility. Alternatively or additionally, a composite evaluation of utility may account for more than one aspect of apparent relevance, such as by indicating more than one pathology of interest.

It deserves emphasis that in such embodiments as that of FIG. 5, an event can occur “in response to” one or more of a prior or contemporaneous measurement, decision, transition, circumstance, or other determinant. Any such event may likewise depend upon one or more other prior, contemporaneous, or potential determinants, in various implementations as taught herein. In other words, such events can occur “in response to” one or more earlier (enabling) events as well as to a later (triggering) event in some contexts.

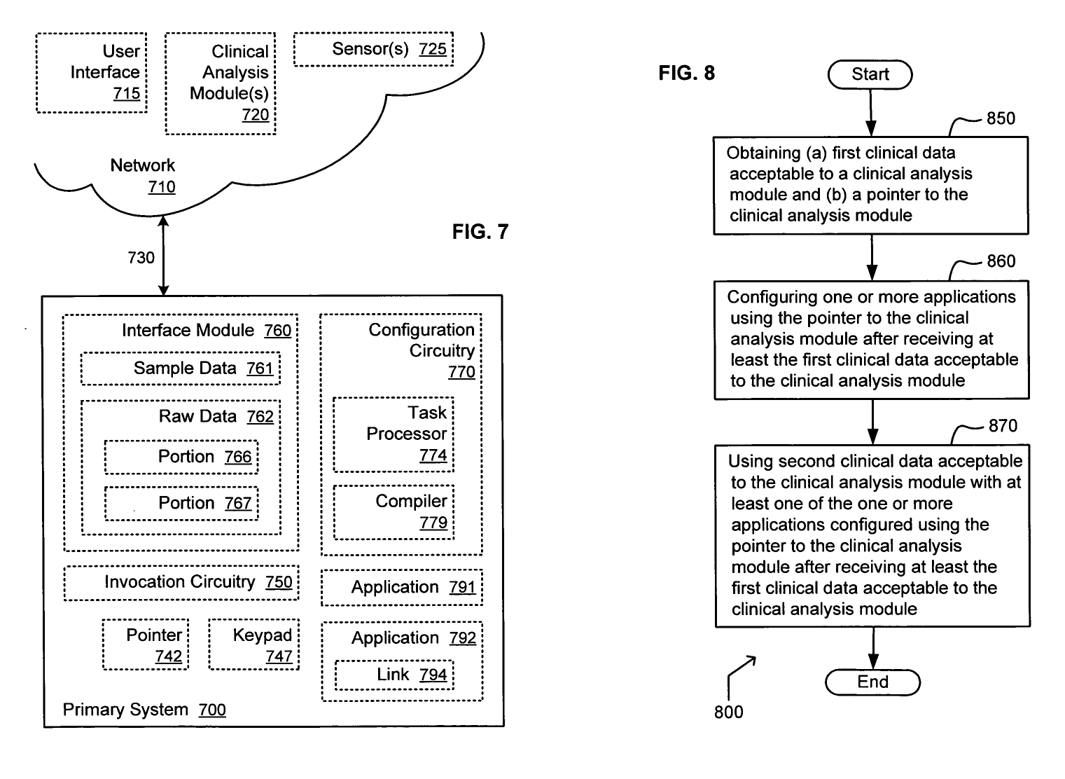

With reference now to FIG. 7, shown is an example of a system that may serve as a context for introducing one or more processes and/or devices described herein. As shown primary system 700 can include one or more instances of pointers 742, keypads 747, invocation circuitry 750, interface modules 760, configuration circuitry 770, or applications 791, 792 (optionally with links 794 as described below). Interface module 760 can include one or more instances of sample data 761, portions 766, 767 of raw data 762, or the like. Configuration circuitry 770 can include one or more instances of task processors 774 or compilers 779. Primary system 700 is operable to communicate with network 710 via network interface 730; and network 710 can include one or more instances of user interfaces 715, clinical analysis modules 720, or sensors 725.

With reference now to FIG. 8, there is shown a high-level logic flow 800 of an operational process. Flow 800 includes operation 850—obtaining (a) first clinical data acceptable to a clinical analysis module and (b) a pointer to the clinical analysis module (e.g. interface module 760 receiving at least sample data 761 and pointer 742). For example, such pointers can identify or otherwise permit access to one or more clinical analysis modules 720 available remotely (e.g. via link 794) or locally (e.g. by being downloaded or otherwise linked to application 792). Alternatively or additionally, the data or pointer can be obtained from within primary system 700, such as via keypad 747. In some embodiments, the clinical data may include data (e.g. portion 767) unacceptable to one or more of the clinical analysis modules 720. Alternatively or additionally, such clinical data can be made acceptable to available clinical analysis modules, such as by enabling or upgrading a module at least partly based on descriptive information about sample data 761 (e.g. whether the sample data is image data, where it is from, or who it is about). In some embodiments, the data or pointer can be received across a conduit or other signal bearing medium (e.g. via network interface 730). Optionally, interface module 760 can be configured to apply one or more criteria for determining whether sample data 761 is acceptable to the clinical analysis module(s), as described herein.

Flow 800 further includes operation 860—configuring one or more applications using the pointer to the clinical analysis module after receiving at least the first clinical data acceptable to the clinical analysis module (e.g. configuration circuitry 770 generating or adapting application 791 or application 792, including or otherwise using one or more instances of the above-described pointers or link 794 after completing operation 850). Configuration circuitry 770 can (optionally) include task processor 774 or compiler 779, for example, capable of performing such configuration. In some variants, of course, operation 850 can repeat or resume after operation 860 begins, such as by user interface 715 later receiving some other such pointer(s) or some additional increment, type, or other aspect of data portion 766 (from an operator, e.g.).

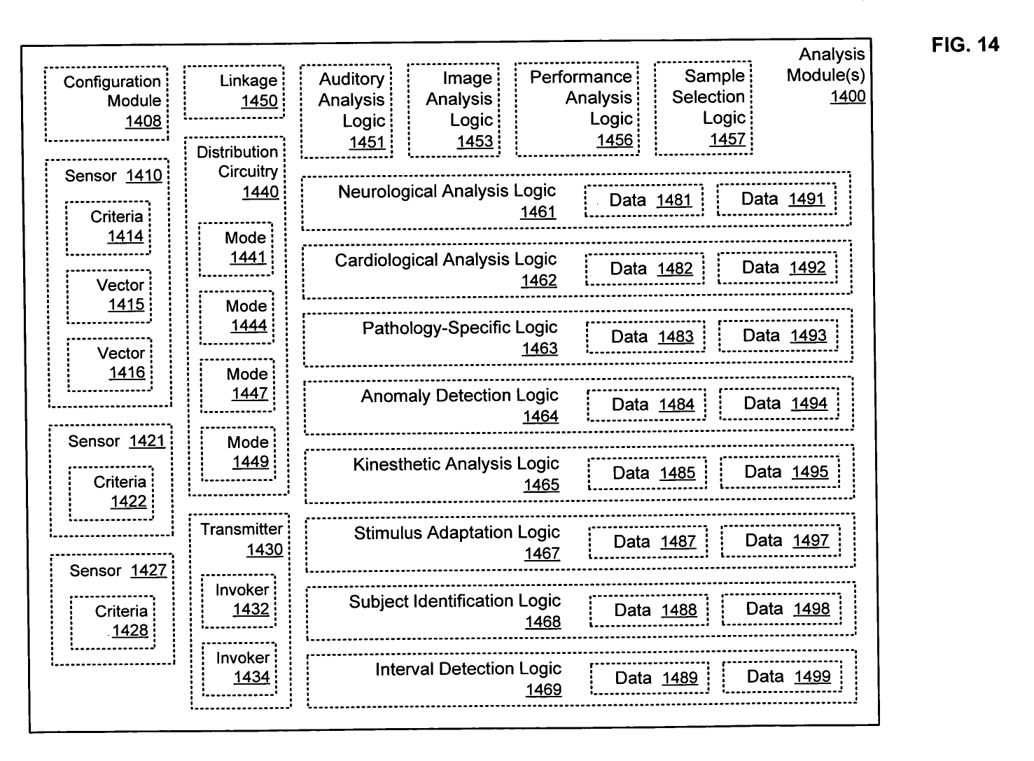

Flow 800 further includes operation 870—using second clinical data acceptable to the clinical analysis module with at least one of the one or more applications configured using the pointer to the clinical analysis module after receiving at least the first clinical data acceptable to the clinical analysis module (e.g. invocation circuitry 750 causing application 791 to use one or more of clinical analysis modules 720 upon data portion 766). In some variants in which data portion 766 includes optical data, for example, invocation circuitry 750 can transmit or identify data portion 766 or the like to image analysis logic as described herein. See, e.g., the discussion of FIG. 14 below. Alternatively or additionally, this can occur before or during operation 860, such as in response to interface module 760 determining that an earlier value of pointer 742 is suitable for some or all types of clinical data arriving at interface module 760.

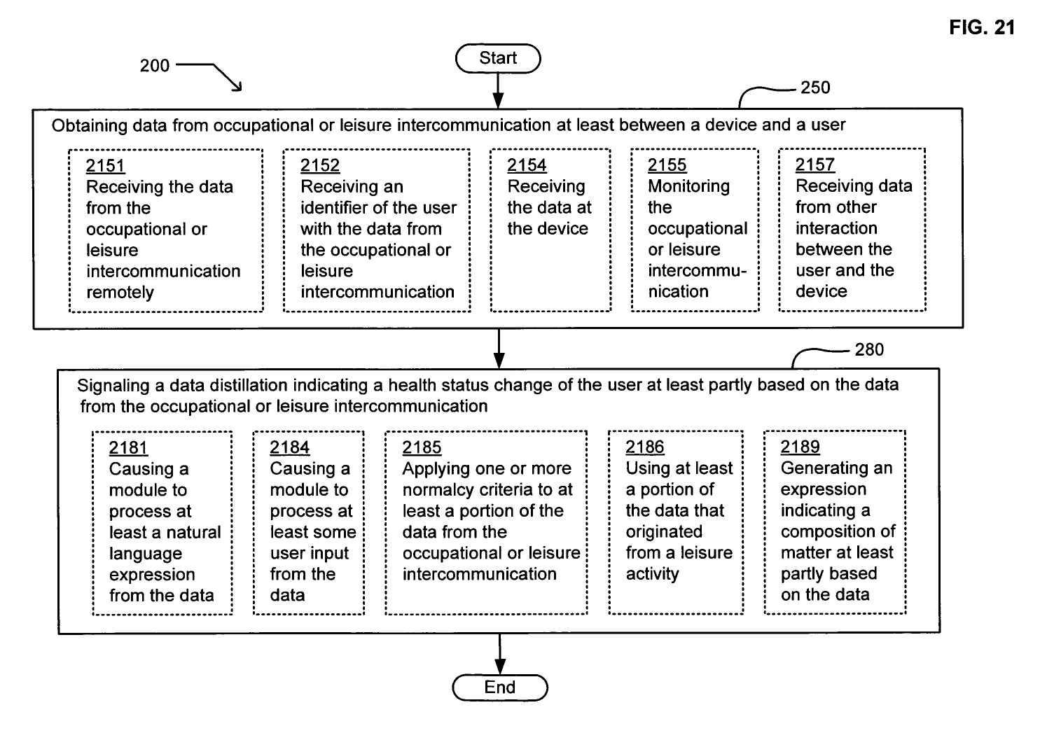

In some embodiments, flow 800 can enable getting some sample data and access to a diagnostic module (at operation 850), installing the diagnostic module or otherwise configuring it for later inputs like the sample data (at operation 860), and causing the diagnostic module to run on other data, such as for monitoring users (at operation 870) as described herein. This can be particularly useful, for example, in situations in which a type or other context of raw data 762 from one or more sensor(s) 725 is not well understood. See, e.g., variants of flow 200 described below in relation to FIGS. 21-23.

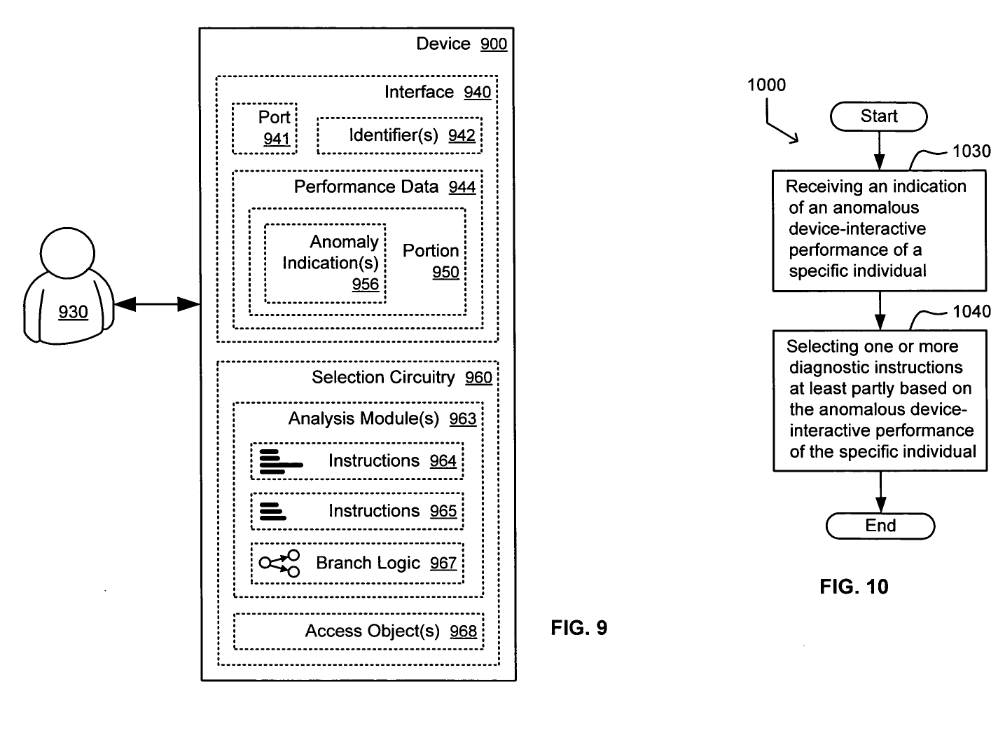

With reference now to FIG. 9, shown is an example of a system that may serve as a context for introducing one or more processes and/or devices described herein. As shown device 900 can optionally observe or otherwise communicate with individual 930. Device 900 can include one or more instances of interfaces 940 or selection circuitry 960. Interface 940 can include one or more instances of port(s) 941, identifiers 942, or performance data 944 (such as anomaly indications 956 or other portions 950, e.g.). Selection circuitry 960 can include one or more instances of analysis modules 963 or access objects 968. Such an analysis module 963 can contain one or more instances of instructions 964, 965 or branch logic 967.

With reference now to FIG. 10, there is shown a high-level logic flow 1000 of an operational process. Flow 1000 includes operation 1030—receiving an indication of an anomalous device-interactive performance of a specific individual (e.g. interface 940 receiving an indication of or otherwise detecting a task or other activity that somehow deviates from normalcy in its manner, efficiency, outcome, degree, occurrence, timing, or other aspect). This can occur in the context of FIG. 1, for example, by receiving raw data 182 or output 188 showing that leisure intercommunication between device 110 and user 130 indicates a level of effectiveness that differs substantially from a prior observed range of behavior for user 130, as measured by some suitable measure of productivity or other performance. (Those skilled in the art will recognize that a “substantial” difference can reasonably comprise a deviation of at least X %, for example, with X typically in a range of 5 to 50 for contexts as described herein.)

Flow 1000 further includes operation 1040—selecting one or more diagnostic instructions at least partly based on the anomalous device-interactive performance of the specific individual (e.g. selection circuitry 960 selecting special-purpose instructions 964 for analyzing data relating to specific individual 930 in a batch process using one or more anomaly indication(s) 956 or some other apparently-relevant portion 950 of performance data 944). Such a selection can be appropriate, for example, in response to an attribute of some performance data in response to an activity status change of the user's application program (according to variants of flow 600 described herein, for example), in response to data surreptitiously captured (according to variants of flow 400 described herein, for example), or the like.

In some embodiments, “diagnostic” instructions can include special-purpose device instruction sequences directly or automatically enabling or otherwise able to cause an analysis of one or more user data or the like as described herein. Alternatively or additionally, a diagnostic instruction can be an oral or written instruction given to a patient or other caregiver, for example, prompting an action or omission primarily to facilitate a diagnostic test. Moreover, some “diagnostic” instructions can include software of which at least a portion prompts or guides such an action or omission.

With reference now to FIG. 11, shown is an example of a system that may serve as a context for introducing one or more processes and/or devices described herein. As shown system 1100 includes at least one primary system 1130 operable to communicate with several parties 1101, 1102, 1103, 1104, 1105 via respective linkages 1111, 1112, 1113, 1114, 1115. For example, linkage 1111 can be implemented via device 1181, which can also include screen A 1151 or other decision circuitry 1141. As shown, device 1182 can optionally observe or otherwise communicate with one or more of party 1101 (directly as shown, e.g.), party 1102, or primary system 1130. Primary system 1130 can include one or more instances of ports 1131, 1132, 1133, screen B 1152 or other decision circuitry 1142, screen C 1153 or other decision circuitry 1143, health-status-indicative updates 1170, irrelevant data 1177, medical history 1178, or the like. Health-statu-indicative updates 1170 can include one or more instances of data 1171, 1172 or recent diagnoses 1173. Primary system can also likewise be operable to access screen D 1154 or other external circuitry 1190 via linkage 1116. In some variants, one or more of the linkages 1111-1116 shown are wireless or otherwise direct (e.g. via a passive-media signal path).

With reference now to FIG. 12, there is shown a high-level logic flow 1200 of an operational process. Flow 1200 includes operation 1250—signaling a decision whether to notify a first party at least by applying a first screen to one or more health-status-indicative updates relating to a second party (e.g. some of decision circuitry 1141, 1142, 1143 implementing or otherwise signaling a decision in response to one or more screens 1151-1153 applied to update data relating to the “second” party, of whether or when to notify the “first” party). The “second” party in this context can include one or more subjects of observation such as parties 1101, 1102. The “first” party in this context can include one or more other parties 1101-1105 to be notified. The update data can include specific health-status-indicative updates 1170 as well as other information such as irrelevant data 1177 or medical history 1178 relating to such a patient. Such health-status-indicative updates 1170 can be configured or otherwise designated for distillation as described in variants of flows 200 or 600 described below, for example, such as by retaining apparently-more-relevant data 1171 or by filtering out apparently-less-relevant data 1172.

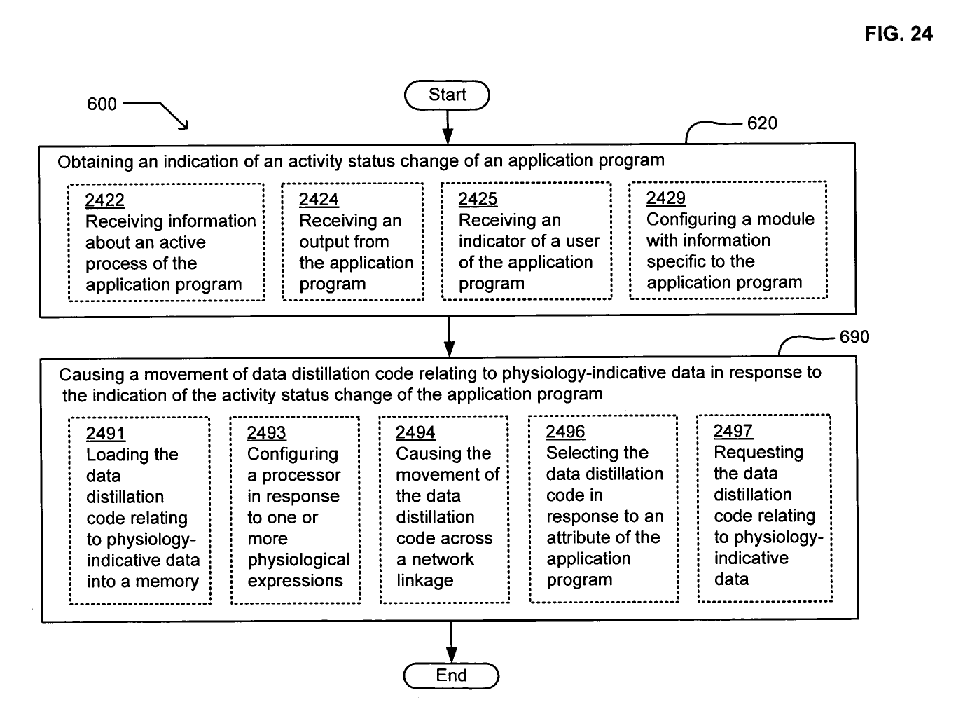

Flow 1200 further includes operation 1280—signaling a decision whether to notify a third party at least by applying a second screen to the one or more health-status-indicative updates relating to the second party (e.g. other decision circuitry 1142, 1143 or external circuitry 1144 signaling a decision resulting from applying one or more other screens 1152-1154 at least to some of the update information relating to the “second” party). The decision can specify how, when, or whether the “third” party (e.g. parties 1101, 1105) is or was notified. The content of such notifications can, for example, can include one or more distillations relating to health-status-indicative data or other information as described herein. See, e.g., FIGS. 24-29 and accompanying descriptions below.

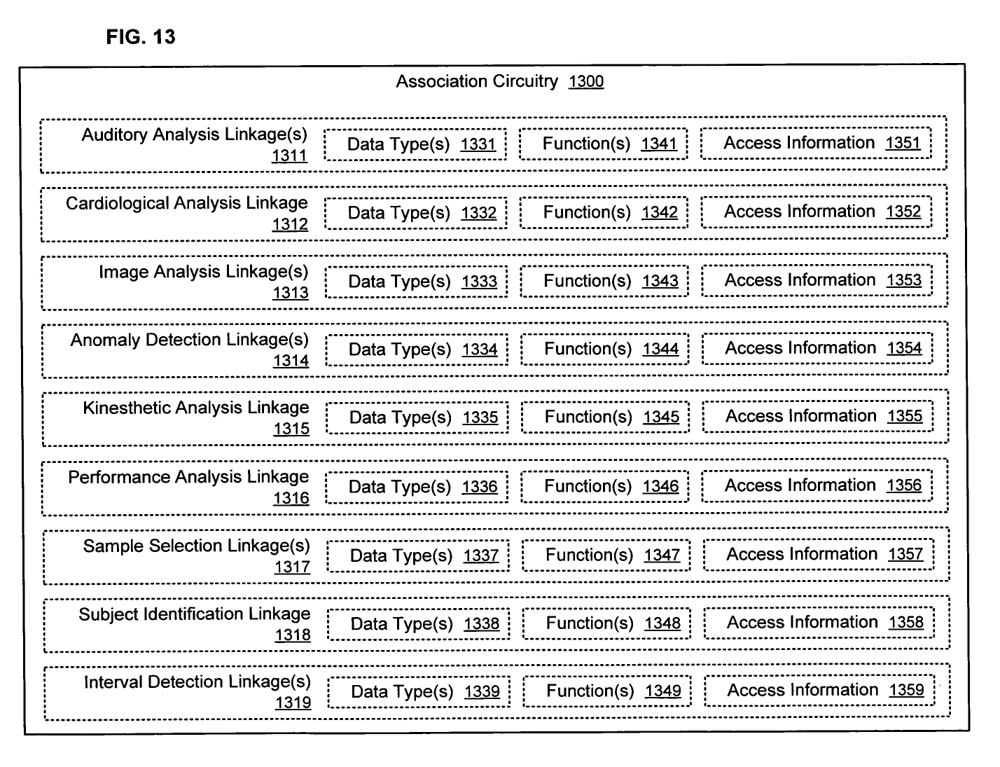

With reference now to FIG. 13, shown is an example of circuitry that may serve as a context for introducing one or more processes and/or devices described herein. As shown association circuitry 1300 can (optionally) include one or more instances of auditory analysis linkages 1311 linking with one or more instances of data types 1331, functions 1341, or access information 1351 described herein in relation to auditory analysis or the like. Association circuitry 1300 can likewise include one or more instances of cardiological analysis linkages 1312 linking with one or more instances of data types 1332, functions 1342, or access information 1352 described herein in relation to cardiological analysis or the like. Association circuitry 1300 can likewise include one or more instances of image analysis linkages 1313 linking with one or more instances of data types 1333, functions 1343, or access information 1353 described herein in relation to image analysis, manipulation, or the like. Association circuitry 1300 can likewise include one or more instances of anomaly detection linkages 1314 linking with one or more instances of data types 1334, functions 1344, or access information 1354 described herein in relation to anomaly detection, analysis, or the like. Association circuitry 1300 can likewise include one or more instances of kinesthetic analysis linkages 1315 linking with one or more instances of data types 1335, functions 1345, or access information 1355 described herein in relation to kinesthetic analysis or the like. Association circuitry 1300 can likewise include one or more instances of performance analysis linkages 1316 linking with one or more instances of data types 1336, functions 1346, or access information 1356 described herein in relation to performance analysis or the like. Association circuitry 1300 can likewise include one or more instances of sample selection linkages 1317 linking with one or more instances of data types 1337, functions 1347, or access information 1357 described herein in relation to sample selection, sample analysis, or the like. Association circuitry 1300 can likewise include one or more instances of subject identification linkages 1318 linking with one or more instances of data types 1338, functions 1348, or access information 1358 described herein in relation to subject identification, comparative analysis, group inclusion, or the like. Association circuitry 1300 can likewise include one or more instances of interval detection linkages 1319 linking with one or more instances of data types 1339, functions 1349, or access information 1359 described herein in relation to interval detection, timing analysis, or the like. Any of such linkages 1311-1319 can, of course, take any of many forms discernable by teachings herein or known in the art, such as by pointers, names, protocols, predetermined structures, hard wiring or coding, or the like. Some varieties or instances of association circuitry can be implemented, for example, in device 110 or primary system 100 of FIG. 1, in handheld or other devices 300 or external module 380 of FIG. 3, in primary system 510 or external system 590 of FIG. 5, in clinical analysis modules 720 or primary system 700 of FIG. 7, in device 900 of FIG. 9, in primary system 1130 or devices 1181, 1182 of FIG. 11, within other modules or media as described herein, or for handling other data as described herein.

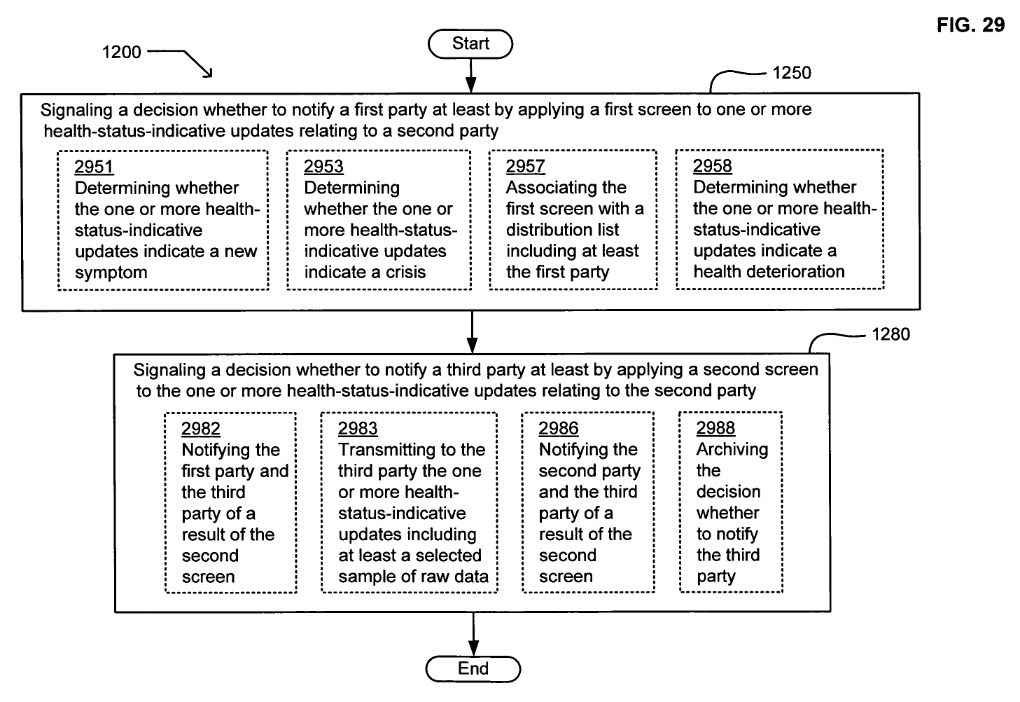

With reference now to FIG. 14, shown is an example of a system that may serve as a context for introducing one or more processes and/or devices described herein. As shown the system can include analysis module(s) 1400 comprising one or more instances of configuration modules 1408, sensors 1410, 1421, 1428, linkages 1450, distribution circuitry 1440, or invokers 1432, 1434 or other transmitters 1430. Sensor 1410 can include one or more criteria such as those described below relating to vectors 1415, 1416 as described below in relation to FIG. 29. Sensors 1421, 1427 can (optionally) each respectively implement one or more criteria 1422, 1428. Linkage 1450 can implement some or all of association circuitry 1300, in some embodiments. Distribution logic 1440 can likewise implement one or more instances of modes 1441, 1444, 1447, 1449 as described below, for example, in relation to FIG. 29.

Analysis module(s) 1400 can further include one or more instances of auditory analysis logic 1451, image analysis logic 1453, performance analysis logic 1456, sample selection logic 1457, neurological analysis logic 1461, cardiological analysis logic 1462, pathology-specific logic 1463, anomaly detection logic 1464, kinesthetic analysis logic 1465, stimulus adaptation logic 1467, subject identification logic 1468, interval detection logic 1469, or the like. At various times and in various embodiments, analysis module(s) 1400 can likewise obtain one or more instances of data 1481, 1482, 1483, 1484, 1485, 1487, 1488, 1489 or data 1491, 1492, 1493, 1494, 1495, 1497, 1498, 1499 respectively as shown. It deserves emphasis that many of these data items can be generated by or otherwise relate to other logic modules than that shown explicitly in FIG. 14. Data 1498 of subject identification logic 1468, for example, can relate just as strongly with one or more of image analysis logic 1456, sample selection logic 1457, neurological analysis logic 1461, or pathology-specific logic 1463.

In some variants of analysis module(s) 1400, some or all of logic 1451, 1453, 1456, 1457, 1461, 1462, 1463, 1464, 1465, 1467, 1468, 1469 can (optionally) be configured automatically, without any end-user input. Alternatively or additionally, some or all of logic 1451, 1453, 1456, 1457, 1461, 1462, 1463, 1464, 1465, 1467, 1468, 1469 can be configured in response to a user-specific goal. Neurological analysis logic 1461, for example, can be configured in response to a request to check a user's reaction time against a nominal standard such as the user's own baseline or that of a population that includes the user.

Alternatively or additionally, some or all of logic 1451, 1453, 1456, 1457, 1461, 1462, 1463, 1464, 1465, 1467, 1468, 1469 can (optionally) be configured to monitor a user or interaction passively—from outside an application or other user function(s) with which a user interacts, for example. In some implementations, such logic can be merely responsive, such as by configuring auditory analysis logic 1451 to be effective for obtaining or retaining a record of vomiting, flatulence, smoking, or other distinguishable audible phenomena susceptible of indicative recordation or other detection sufficient to provide data relating to an individual's health using available system resources. Alternatively or additionally, some or all of logic 1451, 1453, 1456, 1457, 1461, 1462, 1463, 1464, 1465, 1467, 1468, 1469 can be configured as a part of the application or other user function(s) with which the user interacts—such as by adapting stimulus adaptation logic 1467 so that the user can see or hear different stimuli.

Alternatively or additionally, pathology-specific logic 1463 can be chosen or configured to detect, test, infer, predict, or diagnose, and/or to confirm, refute or otherwise test a hypothesis of interest for one or more specific individuals. Such analysis can be performed in relation to participants in a virtual reality environment, for example, measuring reaction times, color perceptions, visual fields, losses in performance when switching tasks, ability to process multiple tasks, short term memory, long term memory, medication side effects, medication efficacy, correlates of medication compliance, or the like as data 1483, 1493.

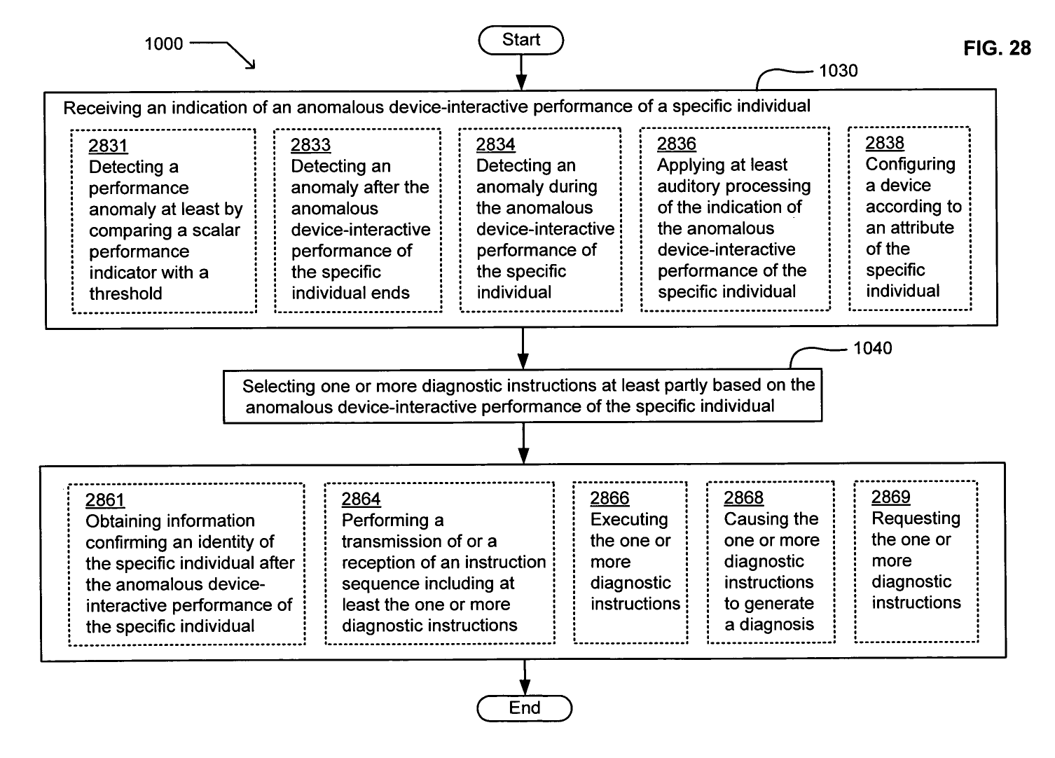

Alternatively or additionally, measurement data 1489 or other inference data 1499 from interval detection logic 1469 can be used by one or more others of logic 1451, 1453, 1456, 1457, 1461, 1462, 1463, 1464, 1465, 1467, 1468. Subject identification logic 1468 can respond to a drastic reaction time improvement, for example, by inferring a likelihood that a new individual is now using a given interface. See FIG. 28.

Alternatively or additionally, anomaly detection logic 1464 can detect normalcy-indicative data 1484 in one, two, or several aspects: reaction time, user effectiveness, user appearance, heart rate or other bioinformatic data, user location, or the like. In some variants, anomaly detection logic 1464 can be configured to analyze raw input data (from data 1484, e.g.) and use it for detecting whether an abnormal level of ambient light or noise or temperature or other attributes exist in a vicinity of an input device (keyboard or other sensor, e.g.). Alternatively or additionally, anomaly detection logic 1464 can be configured to detect anomalies in a correlation or other relationship among two or more measured parameters: a user's apparent capacities or bioinformatic data, times of day, environmental attributes, inputs from a third party (nurse or parent, e.g.), comparisons with historical or other benchmark data, or the like.

Alternatively or additionally, some or all data 1481, 1482, 1483, 1484, 1485, 1487, 1488, 1489 or data 1491, 1492, 1493, 1494, 1495, 1497, 1498, 1499 can give rise to one or more of broadcasting to multiple parties or selective notifications (see FIG. 12, e.g.), sampling or aggregation, real-time processing or other distillation, storage or other follow-up actions, or the like. Some varieties or instances of analysis modules 1400 can be implemented, for example, in device 110 or primary system 100 of FIG. 1, in primary system 510 or external system 590 of FIG. 5, in clinical analysis modules 720 or primary system 700 of FIG. 7, in device 900 of FIG. 9, in primary system 1130 or devices 1181, 1182 of FIG. 11, within other modules or media as described herein, as a stand-alone system, and/or for handling other data as described herein.

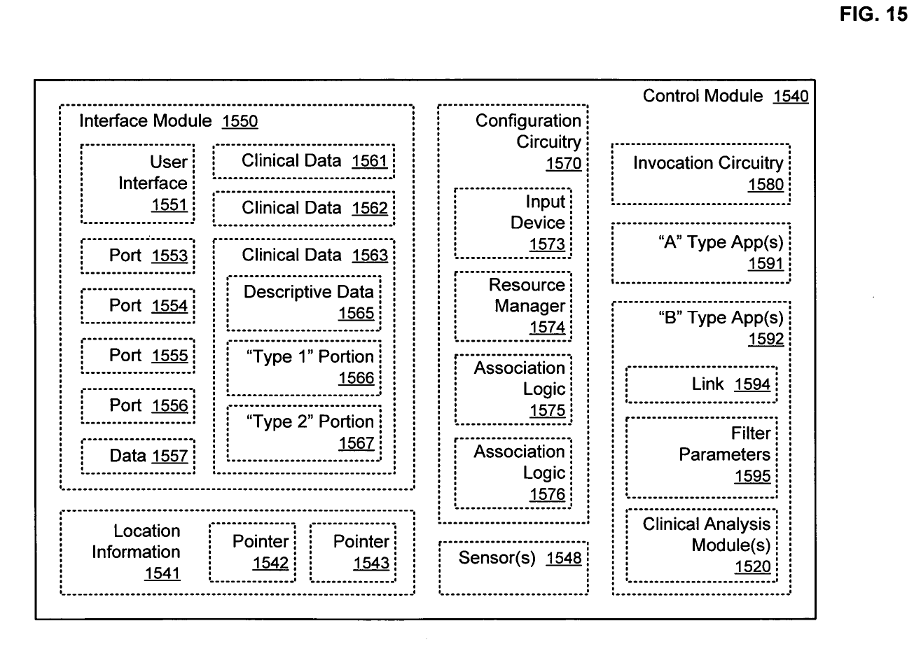

With reference now to FIG. 15, shown is an example of a system that may serve as a context for introducing one or more processes and/or devices described herein. As shown the system includes at least control module 1540 including one or more instances of location information 1541 or pointers 1542, 1543, sensors 1548, interface modules 1550, configuration circuitry 1570, invocation circuitry 1580, “A” type applications 1591, or “B” type applications 1592. Interface module 1550 can, for example, include one or more instances of user interfaces 1551; ports 1553, 1554, 1555, 1556, 1557; or clinical data 1561, 1562, 1563. Clinical data 1563 can include one or more instances of descriptive data 1565, “Type I” portions 1575, or “Type 2” portions 1567. Configuration circuitry 1570 can include one or more instances of input devices 1573, resource managers 1574, or association logic 1575, 1576. “B” type applications 1592 can include one or more instances of links 1594, filter parameters 1595, or clinical analysis modules 1520 such as those of FIG. 14. Some varieties or instances of control module 1500 can be implemented, for example, in device 110 or primary system 100 of FIG. 1, in primary system 510 or external system 590 of FIG. 5, in clinical analysis modules 720 or primary system 700 of FIG. 7, in device 900 of FIG. 9, within other modules or media as described herein, or for handling other data as described herein.

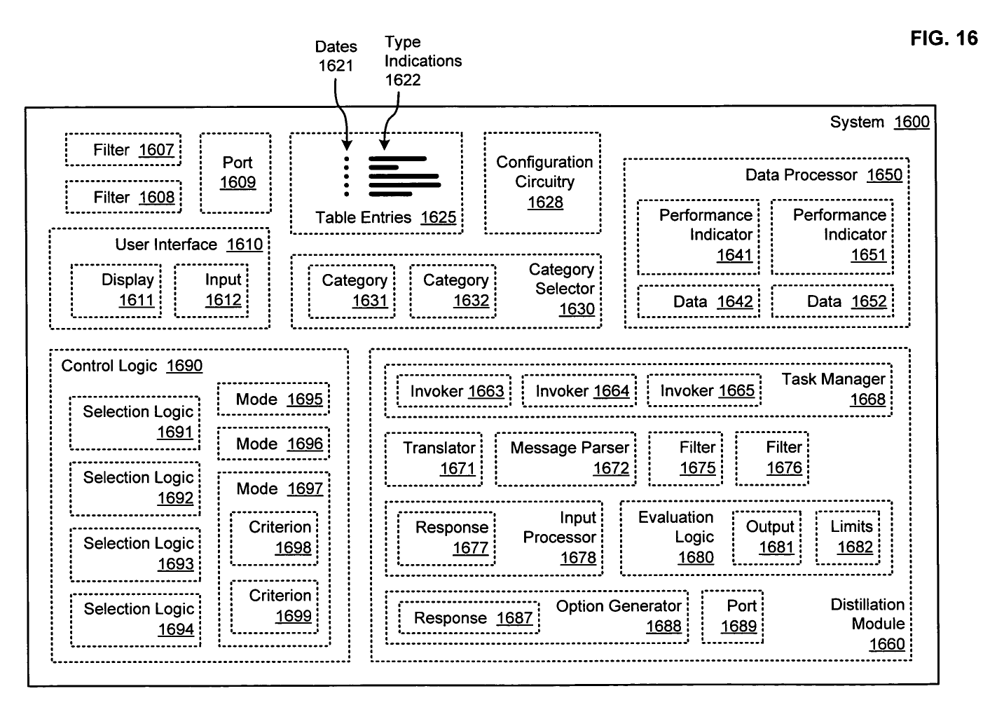

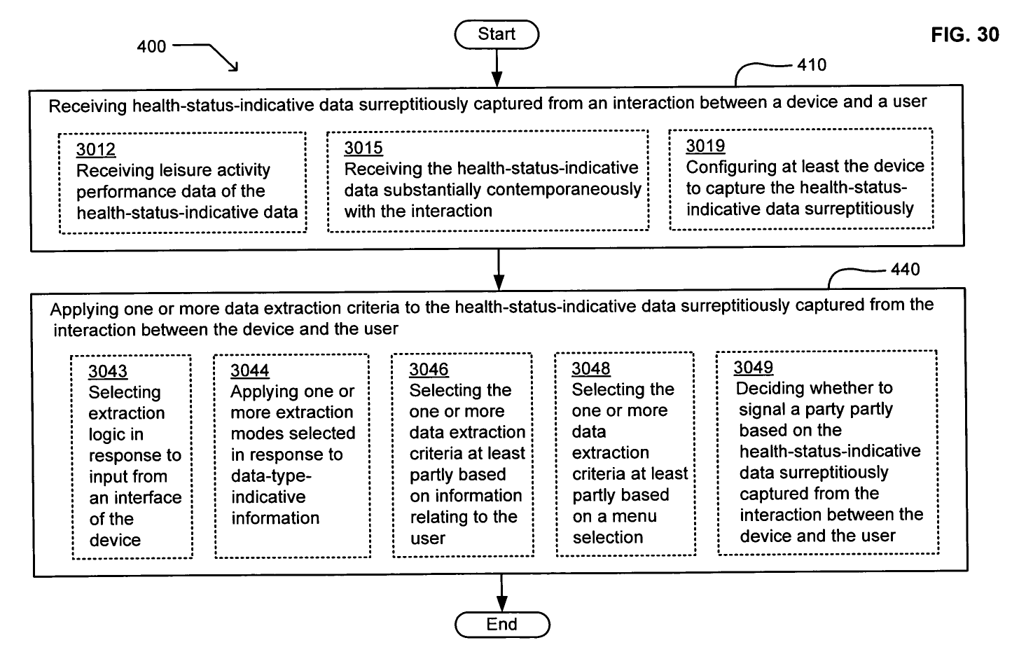

With reference now to FIG. 16, shown is an example of a system that may serve as a context for introducing one or more processes and/or devices described herein. As shown system 1600 can include one or more instances of filters 1607, 1608, ports 1609, user interfaces 1610, table entries 1625 comprising at least dates 1621 and type indications 1622, configuration circuitry 1628, category selectors 1630 operable for selecting one or more categories 1631, 1632, data processors 1650, distillation modules 1660, or control logic 1690. Data processor 1650 can handle or otherwise include one or more instances of performance indicators 1641, 1651 or data 1642, 1652. Control logic 1690 can (optionally) include one or more instances of selection logic 1691, 1692, 1693, 1694 or one or more modes 1695, 1696, 1697. At least mode 1697 relates to one or more criteria 1698, 1699 as described below with reference to FIG. 30.

As shown, distillation module 1660 can (optionally) include one or more instances of invokers 1663, 1664, 1665 in one or more task managers 1668, translators 1671, message parsers 1672, filters 1675, 1676, input processors 1678, evaluation logic 1680, option generators 1688, or ports 1689. As described below with reference to FIG. 21, input processor 1678 can optionally obtain one or more responses 1677 as can option generator 1688 obtain one or more responses 1688. Likewise evaluation logic 1680 can obtain one or more instances of outputs 1681 or limits 1682. Some varieties or instances of system 1600 can be implemented, for example, in device 110 or primary system 100 of FIG. 1, in handheld or other devices 300 or external module 380 of FIG. 3, in clinical analysis modules 720 or primary system 700 of FIG. 7, in device 900 of FIG. 9, in primary system 1130 or devices 1181, 1182 of FIG. 11, within other modules or media as described herein, or for handling other data as described herein.

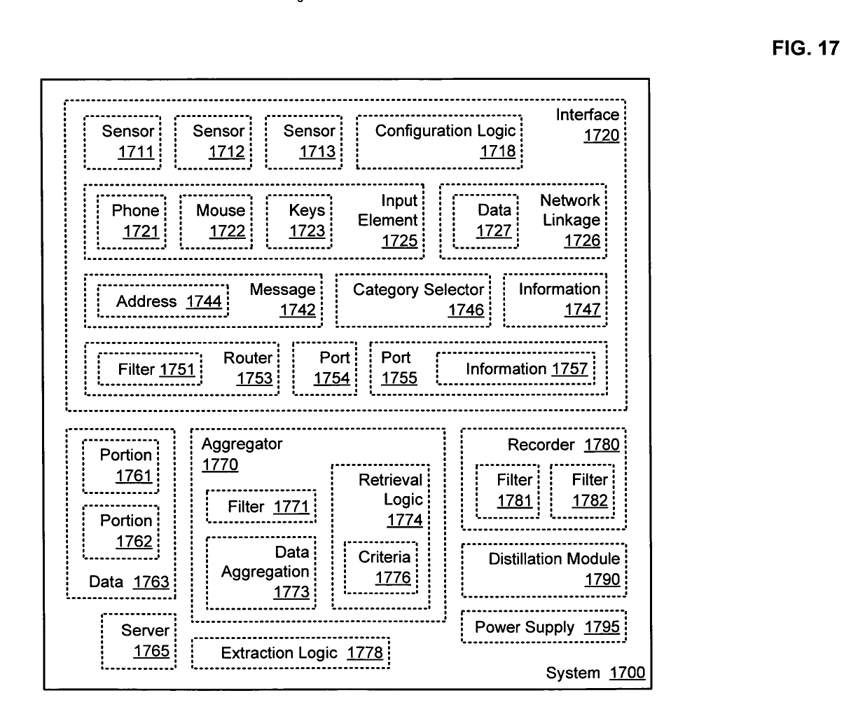

With reference now to FIG. 17, shown is an example of a system that may serve as a context for introducing one or more processes and/or devices described herein. As shown system 1700 can include one or more instances of interfaces 1720, portions 1761, 1762 of data 1763, servers 1765, aggregators 1770, evaluation logic 1778, recorders 1780, distillation modules 1790, or power supplies 1795. Recorder 1780 can, for example, include one or more instances of filters 1781, 1782. Aggregator 1770 can include one or more instances of filters 1771, data aggregations 1773, or retrieval logic 1774 (optionally with one or more criteria 1776). Interface 1720 can include one or more instances of sensors 1711, 1712, 1713; configuration logic 1718; various types of phones 1721, mice 1722, keys 1723, or other input elements 1725; network linkages 1726 for handling data 1727; messages 1744 (optionally with one or more addresses 1744 or the like); category selectors 1746; information 1747; routers 1753 (optionally with one or more filters 1751); or ports 1754, 1755 (optionally with other information 1757). Some varieties or instances of system 1700 can be implemented, for example, in device 110 or primary system 100 of FIG. 1, in handheld or other devices 300 or external module 380 of FIG. 3, in clinical analysis modules 720 or primary system 700 of FIG. 7, in device 900 of FIG. 9, in primary system 1130 or devices 1181, 1182 of FIG. 11, within other modules or media as described herein, or for handling other data as described herein.

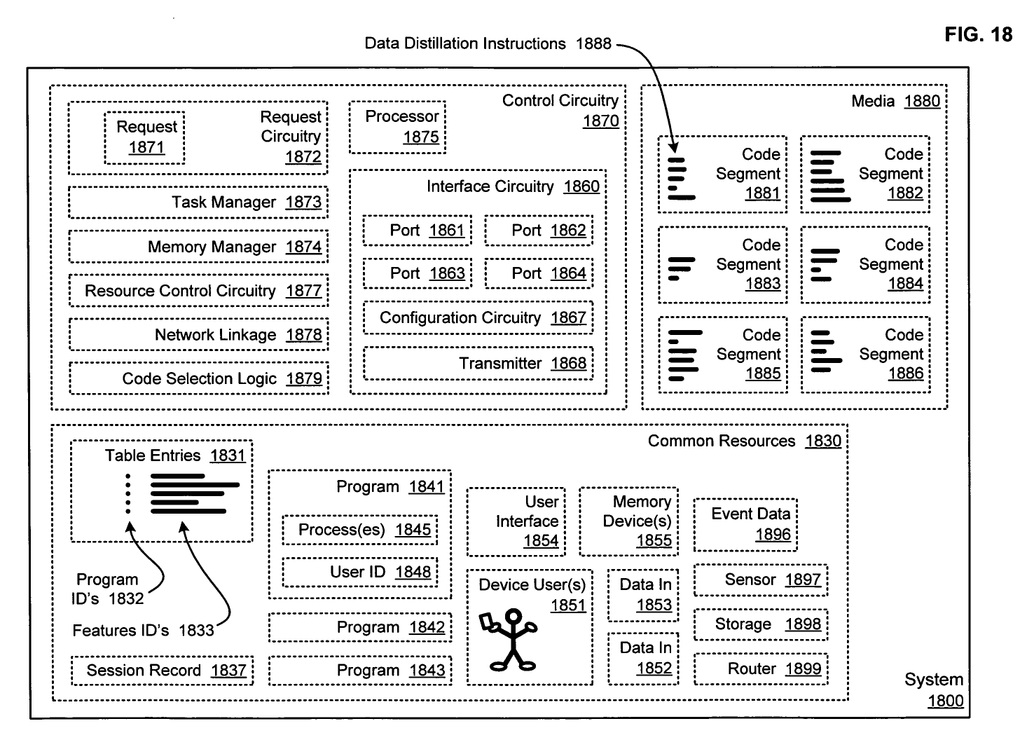

With reference now to FIG. 18, shown is an example of a system that may serve as a context for introducing one or more processes and/or devices described herein. As shown system 1800 can (optionally) include one or more instances of control circuitry 1870, media 1880, or common resources 1830. Control circuitry 1870 can include one or more instances of interface circuitry 1860, request circuitry 1872 for handling requests 1871, task managers 1873, memory managers 1874, processors 1875, resource control circuitry 1877 (including or otherwise accessing resources), network linkages 1878, code selection logic 1879. Interface circuitry 1860 can include one or more of ports 1861, 1862, 1863, 1864; configuration circuitry 1867; or transmitters 1868. One or more media 1880 can each include one or more instances of data distillation instructions 1888 or the like in instruction sequences or other code segments 1881, 1882, 1883, 1884, 1885, 1886. Common resources 1830 can include one or more instances of table entries 1831 comprising one or more program identifiers linked with at least feature identifiers 1833 (see FIG. 24); session records 1837; programs 1841, 1842, 1843 optionally with processes 1845 linking with user identifier(s) 1848; user interfaces 1854 and device users 1851; memory devices 1855; data in 1852, 1853; event data 1896; sensors 1897; storage 1898; or routers 1899. Some varieties or instances of system 1800 can be implemented, for example, in device 110 or primary system 100 of FIG. 1, in primary system 510 or external system 590 of FIG. 5, in clinical analysis modules 720 or primary system 700 of FIG. 7, in device 900 of FIG. 9, within other modules or media as described herein, as a hand-held or other portable system, or for handling other data as described herein.

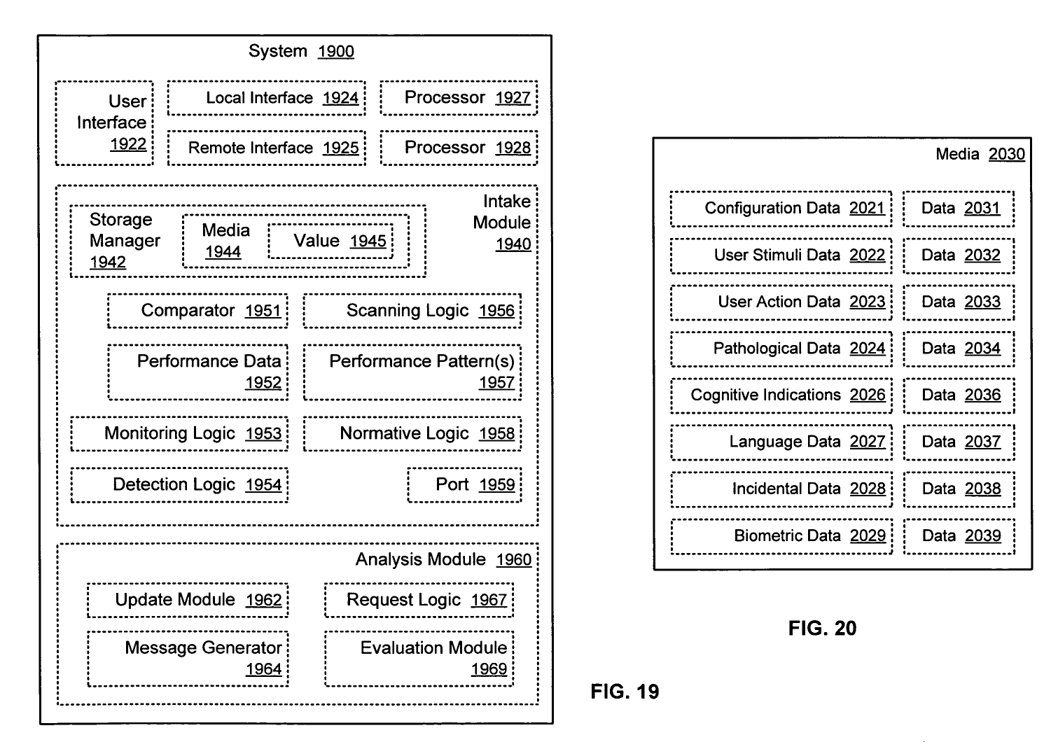

With reference now to FIG. 19, shown is an example of a system that may serve as a context for introducing one or more processes and/or devices described herein. As shown system 1900 can include one or more instances of interfaces 1922, 1924, 1925; processors 1927, 1928; intake modules 1940; or analysis modules 1940. As shown, intake module 1940 can include one or more instances of storage manager 1942 (with values 1945 in one or more media 1944, e.g.), comparators 1951, performance data 1952, monitoring logic 1953, detection logic 1954, scanning logic 1956, performance patterns 1957, normative logic 1958, or ports 1959. Analysis logic can likewise (optionally) include one or more instances of update modules 1962, message generators 1964, request logic 1967, or evaluation modules 1969. Some varieties or instances of system 1900 can be implemented, for example, in device 110 or primary system 100 of FIG. 1, in primary system 510 or external system 590 of FIG. 5, in clinical analysis modules 720 or primary system 700 of FIG. 7, in device 900 of FIG. 9, in primary system 1130 or devices 1181, 1182 of FIG. 11, within other modules or media as described herein, or for handling other data as described herein.

With reference now to FIG. 20, shown is an example of one or more tangible and/or physical media that may serve as a context for introducing one or more processes and/or devices described herein. As shown media 2030 can include one or more instances of configuration data 2021, user stimuli data 2022, user action data 2023, pathological data 2024, cognitive indications 2026, language data 2027, incidental data 2028, biometric data 2029, or other data 2031, 2032, 2033, 2034, 2035, 2036, 2037, 2038, 2039 as described below. Some varieties or instances of media 2030 can be implemented, for example, in device 110 or primary system 100 of FIG. 1, in primary system 510 or external system 590 of FIG. 5, in clinical analysis modules 720 or primary system 700 of FIG. 7, in device 900 of FIG. 9, in primary system 1130 or devices 1181, 1182 of FIG. 11, or for handling other modules or data as described herein.

With reference now to FIG. 21, there are shown several variants of the flow 200 of FIG. 2. Operation 250—obtaining data from occupational or leisure intercommunication at least between a device and a user—may include one or more of the following operations: 2151, 2152, 2154, 2155, or 2157. Operation 280—signaling a data distillation indicating a health status change of the user at least partly based on the data from the occupational or leisure intercommunication—may include one or more of the following operations: 2181, 2184, 2185, 2186, or 2189.

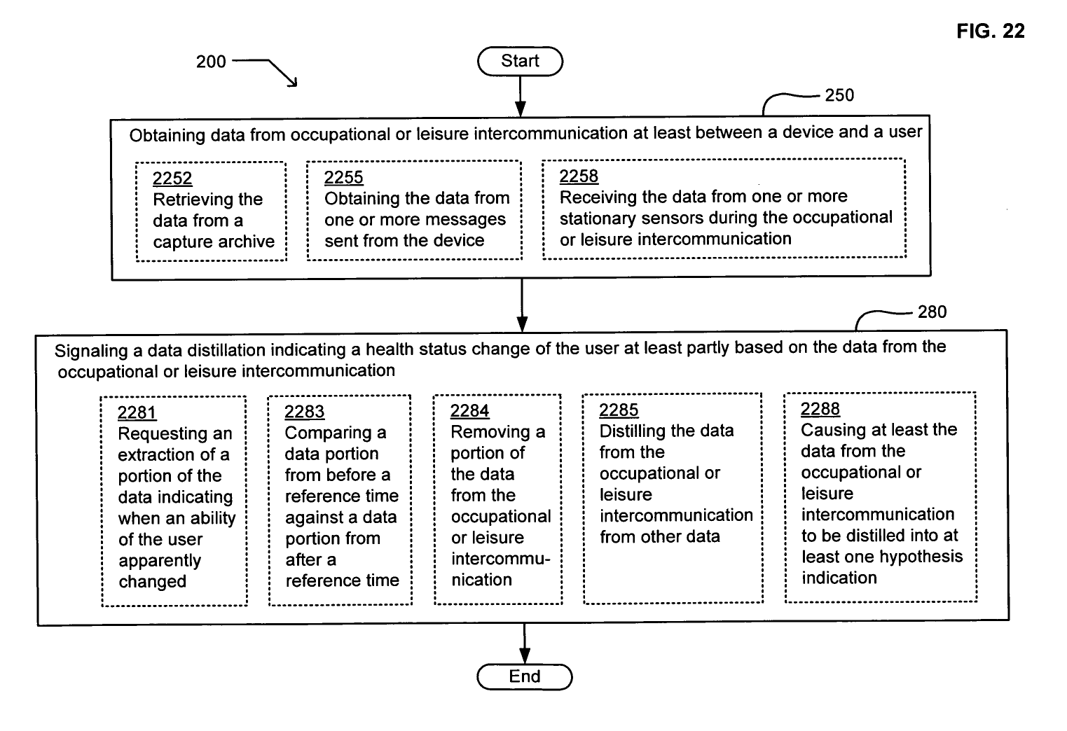

Operation 2151 describes receiving the data from the occupational or leisure intercommunication remotely (e.g. network linkage 171 receiving portions or other indications of intercommunication 135 between user 130 and device 110 as raw data 182). This can occur, for example, in embodiments in which interface module 170 performs operation 250 and in which distillation module 180 performs operation 280. Alternatively or additionally, instances of operation 250 can be performed by embodiments of leisure interfaces 111, occupational interfaces 112, or sensors 113. Likewise some variants of operation 280 can be performed by some implementations of leisure interface 111, occupational interface 112, or other modes of signaling a distillation as described herein. See FIGS. 22-30.

Operation 2152 describes receiving an identifier of the user with the data from the occupational or leisure intercommunication (e.g. server 1765 receiving a text message 1742 or the like containing the sender or recipients' address 1744). A user reading messages or composing sentences steadily and consistently more slowly over a course of months or years may be manifesting a vision, cognitive, or psychological health status change worthy of investigation, for example. In some variants described herein, data can be aggregated from activities of more than one user and compared or otherwise distilled.

Operation 2154 describes receiving the data at the device (e.g. microphone or other phone 1721, mouse 1722, keys 1723, or other input elements 1725 of device 110 receiving user input as a part of the intercommunication). This can occur, for example, in embodiments in which device 110 include one or more instances of system 1700. In a context in which data processing retention is burdensome, in some embodiments one side of the intercommunication (from device 110 to user 130, e.g.) can optionally be ignored.

Operation 2155 describes monitoring the occupational or leisure intercommunication (e.g. one or more of sensors 1712, 1713 detecting intercommunication 135 with occupational interface 112 or some other device in a workplace). This can occur, for example, in embodiments in which device 110 includes one or more instances of systems 1700, with or without any implementation of primary system 100.

Operation 2157 describes receiving data from other interaction between the user and the device (e.g. port 1755 receiving global positioning or other information 1757 from the device, such as to indicate that the user apparently moved the device or otherwise changed its status other than by “intercommunication” with the device). In some contexts, such data from other interaction or other intercommunications can provide contextual information helpful to a data analyst trying to diagnose the apparent health status change or otherwise interpret a record of the intercommunication.

Operation 2181 describes causing a module to process at least a natural language expression from the data (e.g. invoker 1663 causing message parser 1672 to determine whether user 130 is saying something health related). Alternatively or additionally, optical character recognition can be invoked for counting typos or otherwise analyzing text data from a user objectively. Speech recognition can likewise be invoked for detecting slurred speech, long pauses, or other objectively detectable phenomena that can indicate a drug overdose or other health status change of user 130. Such variants can optionally be used with translator 1671 (Spanish to English, e.g.) operable for making a user's speech accessible to a clinical analysis module or caregiver in some embodiments.

Operation 2184 describes causing a module to process at least some user input from the data (e.g. invoker 1664 causing input processor 1678 to interpret keystrokes, sounds, gestures, or the like as an affirmative or negative response 1677). Alternatively or additionally, response 1677 can include one or more instances of location information (via mouse 1722, e.g.), user selections, responses to diagnostic stimuli (as data 1487 from stimulus adaptation logic 1467), or the like.

Operation 2185 describes applying one or more normalcy criteria to at least a portion of the data from the occupational or leisure intercommunication (e.g. evaluation logic 1680 comparing a game score or other application output 1681 against one or more normal minimum or maximum limits 1682). One or more such limits can be defined for a particular user or group of users, for example, dependent upon age, experience at the task, past performance, or the like. Occupational output for a typist can include a typing rate or error rate against a normal minimum or maximum for that typist or for some population of similar typists.

Operation 2186 describes using at least a portion of the data that originated from a leisure activity (e.g. one or more analysis module(s) 1400 using leisure-type intercommunication data such as that of a user chatting or playing a computer game). In some variants of operation 2086, the same or other module(s) can also analyze occupational-type intercommunication data.

Operation 2189 describes generating an expression indicating a composition of matter at least partly based on the data (e.g. option generator 1688 indicating one or more nutraceuticals or other products sometimes used for the apparent health status change). A user showing signs of insomnia, for example, may trigger a response 1687 including chamomile, Lunesta®, ear plugs, or memory foam products. The list may be sent to the user, to a caregiver, or to other interested parties such as advertisers. In some variants, option generator can merely request such options from a remote source and later provide options from whatever response 1687 is received.

With reference now to FIG. 22, there are shown several variants of the flow 200 of FIG. 2 or 21. Operation 250—obtaining data from occupational or leisure intercommunication at least between a device and a user—may include one or more of the following operations: 2252, 2255, or 2258. Operation 280—signaling a data distillation indicating a health status change of the user at least partly based on the data from the occupational or leisure intercommunication—may include one or more of the following operations: 2281, 2283, 2284, 2285, or 2288.

Operation 2252 describes retrieving the data from a capture archive (e.g. retrieval logic 1774 applying search criteria 1776 to extract location, identity, timing, performance, or the like from data aggregation 1773). This can occur, for example, in embodiments in which one or more instances of aggregators 1770 or interfaces 1610, 1720 perform operation 250, in which one or more instances of distillation modules 1660, 1790 perform operation 280, or in which system 1700 is implemented as a stand-alone system or within an embodiment of system 1600.

Operation 2255 describes obtaining the data from one or more messages sent from the device (e.g. filter 1751 detecting medical terms, stress-indicative terms, or the like in voice or text data 1763). In some embodiments, filter 1751 can be configured to search for a pattern that contains natural language terms or other indications of user excitement or distress, anomalies of volume or pace, user-affecting stimuli such as messages from third parties (or from occupational or leisure software), or the like. Alternatively or additionally, filter 1751 can be configured to include some other data in a vicinity of the pattern, or only those packets or the like that contain the pattern.

Operation 2258 describes receiving the data from one or more stationary sensors during the occupational or leisure intercommunication (e.g. network linkage 1726 receiving data 1727 generated by one or more peripheral devices, ad hoc network nodes, or the other stationary elements containing a sensor or other input). In some variants, the device with which the user interacts can include one or more such stationary sensors. For example, one or more instances of sensors 113 within a vicinity of user 130 (e.g. site 120) can security cameras, microphones, touchscreens, or the like.

Operation 2281 describes requesting an extraction of a portion of the data indicating when an ability of the user apparently changed (e.g. invoker 1665 triggering a remote resource to estimate when or whether the user started to exhibit a symptom based on an available record). The remote resource may be a medical diagnostic program, for example, or a physician with a specialty. The available record can include any communication-indicative or other data potentially indicative of a health status change. The request can be a general request or may otherwise refer to matters other than ability change timing. In some instances a response might indicate that the ability has apparently not changed, or it may only narrow down the interval during which the change apparently occurred.

Operation 2283 describes comparing a data portion from before a reference time against a data portion from after a reference time (e.g. data processor 1650 computing performance indicators 1641, 1651 from data 1642, 1652 for respective periods before and after a regimen change or the like). This can be used as a basic comparator (to indicate an apparent 10% loss in performance, e.g.), for hypothesis testing, or for applying other mathematical models to the data 1642, 1652. The reference time can be provided at a user interface, for example, or can be derived from data 1642, 1652 or other medical history information.

Operation 2284 describes removing a portion of the data from the occupational or leisure intercommunication (e.g. filter 1675 removing an irrelevant or otherwise unwanted portion of any of data 1481-1499 of FIG. 14). This can occur, for example, in embodiments in which one or more interfaces 1610, 1720 perform operation 250, in which distillation module 1660 performs operation 280, and in which distillation module 1660 accesses or otherwise implements analysis module(s) 1400. Such distillations can be performed by selectively retaining portions near an event or time of interest, for example, or by removing noise or the like.

Operation 2285 describes distilling the data from the occupational or leisure intercommunication from other data (e.g. filter 1676 removing data relating to other users or devices, data from other types of interactions, or the like). In some variants, configuration circuitry 1628 can (optionally) configure one or more of filters 1675, 1676 according to specific criteria, such as by favoring those relating to a specific pathology of interest. Alternatively or additionally, data records can be kept and distinguished that each relate to a respective user, with some users having attributes in common (e.g. age, ethnicity, genetic commonality, language, health history, or other group attribute). Such group data can be used for establishing normalcy criteria relating to a specific user/member as described herein, for example.

Operation 2288 describes causing at least the data from the occupational or leisure intercommunication to be distilled into at least one hypothesis indication (e.g. category selector 1630 indicating “respiratory,” “speech,” “cognitive,” “memory,” “vision,” “infection,” “intoxication” or other category 1632 having a possible relation to an apparent health status change indicated by such data). In various embodiments, the hypothesis indication(s) can include a diagnosis, prognosis, recommendation, descriptor, or other possibility of potential use to a user, diagnostician, statistician, caregiver, analyst, or the like.

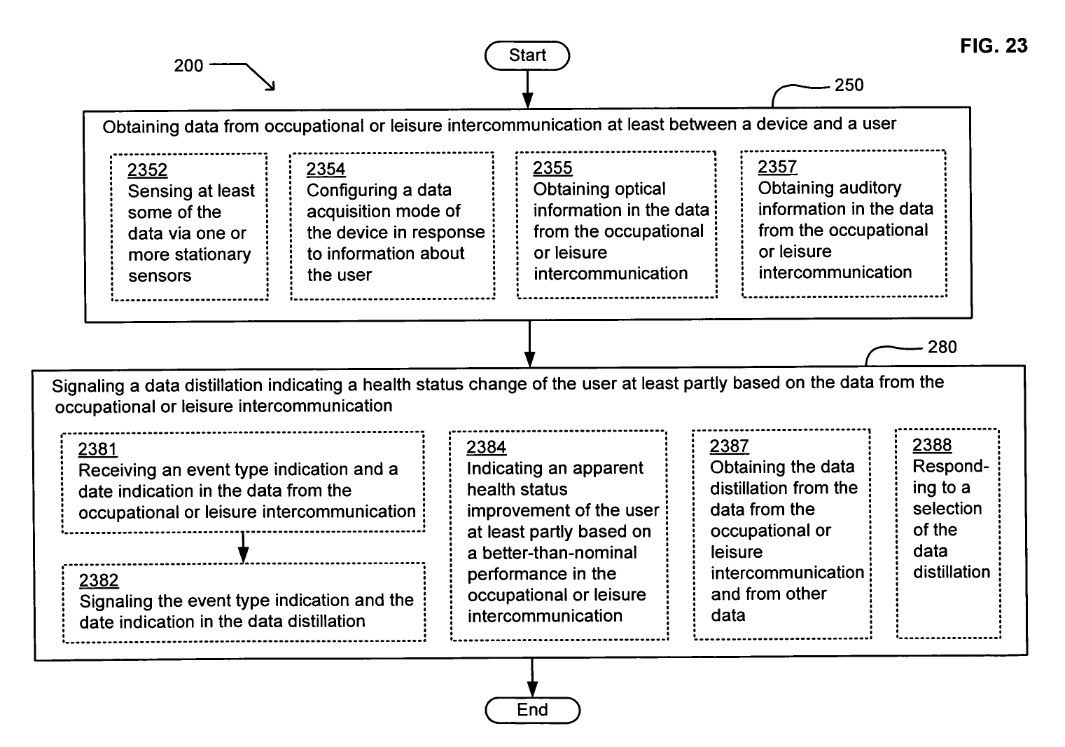

With reference now to FIG. 23, there are shown several variants of the flow 200 of FIG. 2, 21, or 22. Operation 250—obtaining data from occupational or leisure intercommunication at least between a device and a user—may include one or more of the following operations: 2352, 2354, 2355, or 2357. Operation 280—signaling a data distillation indicating a health status change of the user at least partly based on the data from the occupational or leisure intercommunication—may include one or more of the following operations: 2381, 2382, 2384, 2387, or 2388.

Operation 2352 describes sensing at least some of the data via one or more stationary sensors (e.g. sensor 113 or sensor 1712 generating data indicative of communication to or from user 130 via device 110). This can occur, for example, in embodiments in which one or more instances of sensors 113, 1712 are stationary. Alternatively or additionally, system 1700 can be configured with a power supply 1795 operable via an electrical outlet, and optionally including one or more instances of sensors 1711, 1712, 1713, servers 1765, aggregators 1770, recorders 1780, or other elements that can incorporate motors or otherwise consume significant levels of power.

Operation 2354 describes configuring a data acquisition mode of the device in response to information about the user (e.g. category selector 1746 designating signals from microphone or other phone 1721 or the like as data 1493 for pathology-specific logic 1463 in response to a suggestion that a specified user might suffer a disorder with auditory indications). A wide variety of such disorders are well documented and generally susceptible to some degree of direct auditory detection: joint disorders, cardiological disorders, respiratory disorders, or the like. In some instances in which an anomaly is detected for which no category is apparent, aggregator 1770 can respond in a “miscellaneous capture” mode so that some or all data about the anomaly is archived for potential future analysis.

Operation 2355 describes obtaining optical information in the data from the occupational or leisure intercommunication (e.g. image analysis logic 1453 obtaining data from a camera or other sensor 113 in the user's vicinity). This can occur, for example, in embodiments in which interface 1720 and sensor 113 jointly (and each) perform operation 250 and in which one or more instances of aggregators 1770 or distillation modules 1660, 1790 perform operation 280.

Operation 2357 describes obtaining auditory information in the data from the occupational or leisure intercommunication (e.g. auditory analysis logic 1451 obtaining data from a telephonic or other auditory signal associated with the intercommunication). The auditory signal may include speech indicating that the user is having trouble hearing, for example, or may include other sounds in the user's vicinity. In some instances, a variety of phenomena may be relevant to determinations relating to a health status change. Loud noises in a user's environment can contraindicate (or cause) an apparent health status change in some instances, for example. In some variants, sample selection logic preferentially increases an auditory or other sampling rate near anomalies, symptoms identified as significant, or other events that a diagnostician might designate as having a higher-than-nominal utility.

Operation 2381 describes receiving an event type indication and a date indication in the data from the occupational or leisure intercommunication (e.g. port 1609 receiving data including one or more dates 1621 or the like that map to one or more type indications 1622). Such indications can include one or more instances of an interaction type indicator, a user application name, use or productivity statistics, event descriptors, or the like. Such table entry data can, in some instances, also include raw data or other matter of which some might be irrelevant, duplicative, voluminous, or otherwise unwieldy.

Operation 2382 describes signaling the event type indication and the date indication in the data distillation (e.g. filter 1608 removing some or all of the above-referenced “other” matter but passing along some or all of the dates and type indications). In some embodiments, some or all of the resulting data distillation can be presented to one or more interested parties (by display 1611 roughly in real time using flow 1200, e.g.), with or without being captured or archived.

Operation 2384 describes indicating an apparent health status improvement of the user at least partly based on a better-than-nominal performance in the occupational or leisure intercommunication (e.g. leisure interface 111, occupational interface 112, or other occupational or leisure software providing data indicating a sufficiently consistent and/or dramatic improvement in a user's endurance or coordination to support a reasonable inference that the user's physical health has improved). Such an inference is unlikely to be supportable merely by showing improvement within a first few weeks of game play, however, during which time any improvements are at least as attributable to improving skills of the game. Significant improvements at a user's longstanding favorite game, however, can reasonably support an inference that a new medication or exercise regimen is working, especially if corroborated by other signs of improvement. New achievements in productivity with often-used occupational software can likewise provide significant objective evidence of a health status improvement or decline.

Operation 2387 describes obtaining the data distillation from the data from the occupational or leisure intercommunication and from other data (e.g. aggregator 1770 selectively retaining portions of data from the occupational or leisure intercommunications as well as from other interactions, as data aggregation 1773). Alternatively or additionally, data aggregation 1773 can include (the “other”) data from occupational or leisure interactions among other devices and users. In some variants, some or all of the data distillation can relate to extracting a non-probative portion 1761 from data 1763 irrespective of how much of it arises directly from the intercommunication: null event reports, “no change” or default record values, inaudible audio clips, pattern replications, solid black or white images, or the like.

Operation 2388 describes responding to a selection of the data distillation (e.g. sample selection logic 1457 activating one or more other analysis module(s) 1400 in response to one or more system selections). The user or a healthcare provider can select which modules to activate, for example, in response to which one or more portions of data 1481-1499 are selectively obtained, filtered, aggregated, analyzed, selected, evaluated, or otherwise distilled as described herein.

With reference now to FIG. 24, there are shown several variants of the flow 600 of FIG. 6. Operation 620—obtaining an indication of an activity status change of an application program—may include one or more of the following operations: 2422, 2424, 2425, or 2429. Operation 690—causing a movement of data distillation code relating to physiology-indicative data in response to the indication of the activity status change of the application program—may include one or more of the following operations: 2491, 2493, 2494, 2496, or 2497.

Operation 2422 describes receiving information about an active process of the application program (e.g. port 1861 receiving an image name, a process status, a security level, a resource identifier, an owner, a location or the like in relation to one or more processes 1845 of program 1841). Alternatively or additionally, the information can include one or more computed estimates of quantities such as a completion time, a probability, a progress report, a resource usage status, a rate or level, an evaluation or the like relating to any thread, state information, user, task, or other aspect of program 1841.

Operation 2424 describes receiving an output from the application program (e.g. port 1555 receiving an event code or other clinical data 1562 from program 1841). This can occur, for example, in embodiments in which interface circuitry 1860 performs operation 620 (such as by implementing an instance of control module 1540 in interface circuitry 1860), and in which other portions of control circuitry 1870 or common resources 1830 perform operation 690. In some embodiments, the output can be part of what program 1841 presents at user interface 1854 or can include or otherwise reflect data in 1852 received from a patient or remote source indirectly, such as via router 1899.

Operation 2425 describes receiving an indicator of a user of the application program (e.g. Port 1863 receiving user identification 1848, session record 1837, or other data indicative of one or more device users 1851 interacting with program 1841). In some embodiments, the indicator can signal other users interacting with program 1841 or device user(s) 1851 interacting with one or more other programs so as to generate the physiology-indicative data of operation 690.

Operation 2429 describes configuring a module with information specific to the application program (e.g. configuration circuitry 1867 modifying one or more table entries 1831 by including one or more records indicating an object name, path, current status, or other feature identifiers 1833 in association with an identifier 1832 of program 1841 or any of its components). Alternatively or additionally, in some embodiments, configuration circuitry 1867 can make or otherwise signal such configurations in response to changes in files or other records, to messages or other new data, to other signals from active programs, or to other events indicative of program activity. Many such indications are readily available from common programs, depending on the contexts in which they operate.

Operation 2491 describes loading the data distillation code relating to physiology-indicative data into a memory (e.g. memory manager 1874 copying code segment 1881 into or among one or more memory devices 1855). This can occur, for example, in embodiments in which at least control circuitry 1870 performs operation 690, optionally in cooperation with other portions of system 1800. Alternatively or additionally, in an embodiment in which external system 590 of FIG. 5 implements one or more instances of systems 1800, network linkage 1878 can be configured to upload interaction data distillation code 517 and language data 2027 into memory 530 so that the former can be used at least in processing the latter.

Operation 2493 describes configuring a processor in response to one or more physiological expressions (e.g. task manager 1873 queuing processor 1875 to apply a filter comprising code segment 1882 associated with a segment label of “Cardio_{—}33”). Such a physiological expression can optionally include or relate to an body part or other anatomical term, a drug or other regimen feature, a pathology, a medical phenomenon, a surgical procedure, or the like. In some embodiments, the physiological expression or logical equivalent forms a part of a name of a path, a file, a database field name, a process, a variable name, a logical expression, or the like. Alternatively or additionally, processor 1875 can (optionally) be configured by providing it with one or more code segments 1881, 1882, 1883, 1884, 1885, 1886 selected at least partly based on the physiological expression(s).

Operation 2494 describes causing the movement of the data distillation code across a network linkage (e.g. resource control circuitry 1877 requesting that code segment 1883 be transmitted via network linkage 1878). In other instances, memory manager 1874 can be configured to cause code segment 1884 to be copied to or from one or more memory device(s) 1855 remotely via network linkage 1878 or the like. (Alternatively, of course system 1800 can be implemented entirely within one physical site.)

Operation 2496 describes selecting the data distillation code in response to an attribute of the application program (e.g. code selection logic 1879 identifying code segment 1885 with a memory address or other code-identifying information associated with program 1842). Alternatively or additionally, some or all of the code selection information can be received from user interface 1854, table entries 1831, or the like. For example, an operating system may be configured to query or otherwise receive process- or self-identifying information from program 1842.

Operation 2497 describes requesting the data distillation code relating to physiology-indicative data (e.g. request circuitry 1872 responding to physiology-indicative data in 1853 by sending request 1871 that causes code segment 1886 to arrive among a library of distillation code sources). For example, code segment 1886 can be kept in a central or regional server site until a version is needed locally, facilitating the use of up-to-date local code on demand or customized code for distilling the data in 1853.

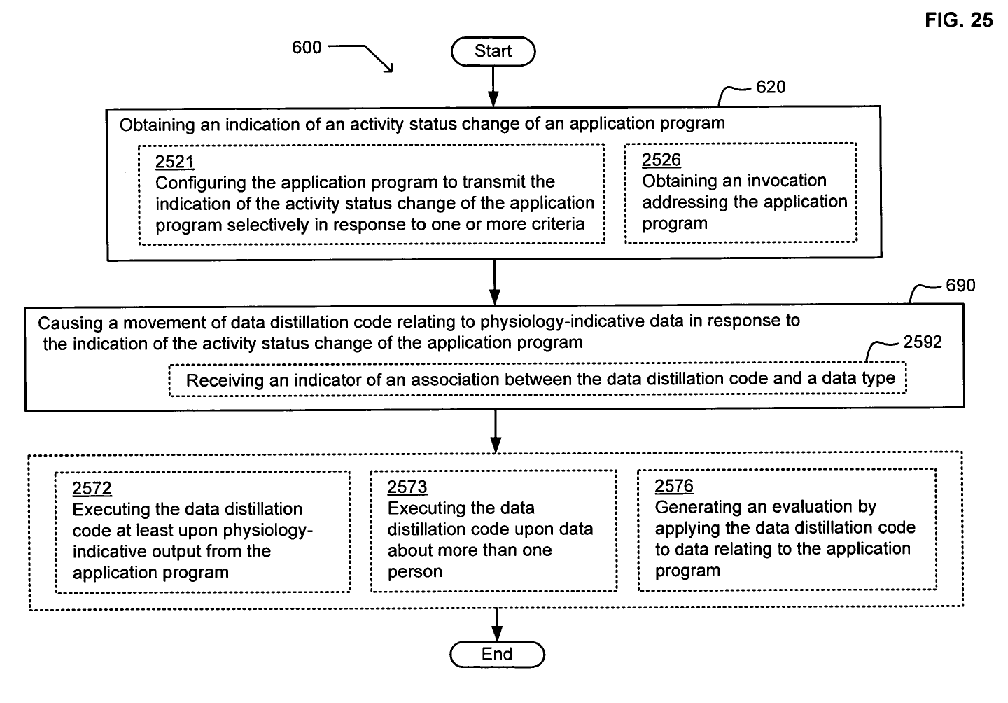

With reference now to FIG. 25, there are shown several variants of the flow 600 of FIG. 6 or 24. Operation 620—obtaining an indication of an activity status change of an application program—may include one or more of the following operations: 2521 or 2526. Operation 690—causing a movement of data distillation code relating to physiology-indicative data in response to the indication of the activity status change of the application program—may include operation 2592. Alternatively or additionally flow 600 may likewise include one or more of operations 2572, 2573, or 2576—each depicted after operation 690 but optionally performed concurrently with it, or in other orders.

Operation 2521 describes configuring the application program to transmit the indication of the activity status change of the application program selectively in response to one or more criteria (e.g. interface circuitry 596 configuring one or more resident programs 594 with one or more criteria 595 operable for deciding when or how to transmit an activation or deactivation signal). In some instances, for example, the one or more criteria 595 can signal a process activation, a rate of progress, an “activate” command, or the like, for example, or a core initialization that includes loading at least part of program 1841. This can occur, for example, in embodiments in which external system 590 performs at least operation 620.

Operation 2526 describes obtaining an invocation addressing the application program (e.g. sensor 1897 receiving an invocation addressing one or more of programs 1841, 1842, 1843). This can occur, for example, in embodiments in which one or more common resources 1830 perform operation 620. In some implementations, such a trigger signal or other invocation can itself indicate a nominal, likely or recent activity status change of the program(s) to which it is addressed.

Operation 2592 describes receiving an indicator of an association between the data distillation code and a data type (e.g. at least some association circuitry 1300 receiving an update or other logic indicating one or more instances of image analysis linkage(s) 1313 linking “jpg,” “mpeg,” or other image-containing data type(s) 1333 with a corresponding reference to data distillation code such as access information 1353). Alternatively or additionally, the association can contain literal implementations of some or all function(s) 1343 appropriate for the associated data type(s) 1333. In some variants, one or more other linkages can be received from association circuitry 1300, for example, by control circuitry 1870 or the like in performing operation 690. In various embodiments, such linkages can include one or more instances of auditory analysis linkage(s) 1311, cardiological analysis linkage(s) 1312, kinesthetic analysis linkage(s) 1315, interval detection linkage(s) 1319, or the like.

Operation 2572 describes executing the data distillation code at least upon physiology-indicative output from the application program (e.g. processor 511 applying optical data distillation code 513 to sample image output from a webcam program, a digital photograph upload utility, an e-mail application, or the like). In some embodiments, user stimuli data 2022 may contain optical images, for example, to show what a user found disturbing or to identify blind portions of a user's field of view (the left side or center, e.g.). User action data 2023 may likewise contain diagnostically relevant optical images, for example, to suggest what a user was doing shortly before suffering a seizure or going into shock. Such images may likewise comprise cognitive indications 2026, for example, showing that a user showed a pattern of drowsiness or agitation. In such cases, the images may likewise contain or accompany timestamps, device identifiers, regimens, or similar incidental data 2028 to describe circumstances under which pathological indications or other physiology-indicative output was captured. Other kinds of program output data 2038 can include data arising from observing patients, data from a user's parent or other caregiver, surreptitious data, or the like as exemplified herein.

Operation 2573 describes executing the data distillation code upon data about more than one person (e.g. processor 511 executing auditory data distillation code 515 upon data from several parties, for example, to establish a normal range of coughing frequencies for a demographic group). Such norms can be useful for initial screening for a wide variety of pathologies. For example, such norms may form a basis for associating a specific user with pathological data 2024 (e.g. detailed data about asthma, bronchitis, and allergic response detection criteria downloaded in response to a preliminary indication of the user's abnormally frequent coughing). Alternatively or additionally, a workforce or other technology-literate community can, in some embodiments, be screened to identify one or more members likely to be suitable for a vaccine or other experimental, preventive or corrective treatment.

Operation 2576 describes generating an evaluation by applying the data distillation code to data relating to the application program (e.g. processor 511 or other circuitry generating one or more data distillations 529 that include a ranking, a level indication, a category, or the like). In some embodiments, the evaluation can be generated using one or more instances of neurological analysis logic 1461, cardiological analysis logic 1462, pathology-specific logic 1463, anomaly detection logic 1464, or any of the other items described herein in relation to data distillation. Alternatively or additionally, the evaluation can be partly or wholly based on one or more instances of configuration data 2021, user stimuli data 2022, user action data 2023, pathological data 2024, language data 2027, incidental data 2028, biometric data 2029 or the like.

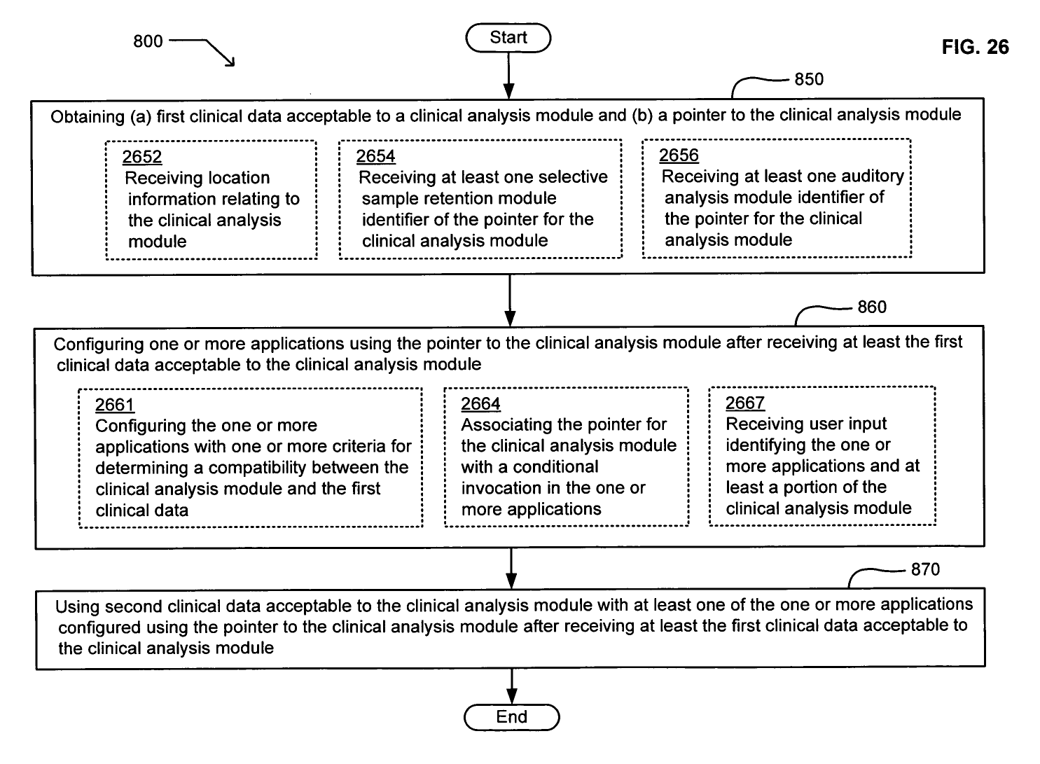

With reference now to FIG. 26, there are shown several variants of the flow 800 of FIG. 8. Operation 850—obtaining (a) first clinical data acceptable to a clinical analysis module and (b) a pointer to the clinical analysis module—may include one or more of the following operations: 2652, 2654, or 2656. Operation 860—configuring one or more applications using the pointer to the clinical analysis module after receiving at least the first clinical data acceptable to the clinical analysis module—may include one or more of the following operations: 2661, 2664, or 2667. Many variants of operation 870—using second clinical data acceptable to the clinical analysis module with at least one of the one or more applications configured using the pointer to the clinical analysis module after receiving at least the first clinical data acceptable to the clinical analysis module—are also provided herein: in which the data is used by a distillation module as described herein in variants of flow 200, for example, or in which performance-indicative data is used according to variants of flow 1000.

Operation 2652 describes receiving location information relating to the clinical analysis module (e.g. port 1553 receiving a path, pointer, or other location information 1541 relating to image analysis logic 1453). This can occur, for example, in embodiments in which interface module 1550 performs operation 850. Alternatively or additionally, location information 1541 can include references to auditory analysis logic 1451, performance analysis logic 1456, or the like. In some embodiments, the location information can be received locally (e.g. user interface 1551 receiving the information from a local operator).

Operation 2654 describes receiving at least one selective sample retention module identifier of the pointer for the clinical analysis module (e.g. port 1554 receiving a logical address or other location information relating to sample selection logic 1457). In some embodiments, such selection logic can comprise selective sample retention logic, indexing or ranking logic, a data compressor or other filter, or other selective mechanism as described with reference to other figures herein.

Operation 2656 describes receiving at least one auditory analysis module identifier of the pointer for the clinical analysis module (e.g. port 1556 receiving a data object name, module name, or other reference data 1557 relating specifically to auditory analysis logic 1451). In some embodiments, one or more ports can likewise receive information sufficient to identify one or more other clinical analysis module(s) 720 or analysis module(s) 1400. Alternatively or additionally, the “first” clinical data can be received from sensor(s) 725 or 1548.

Operation 2661 describes configuring the one or more applications with one or more criteria for determining a compatibility between the clinical analysis module and the first clinical data (e.g. resource manager 1574 providing “B” type app(s) 1592 with file types, protocol version numbers, or the other filter parameters 1595 for generating an indication of whether performance analysis logic 1456 can apparently process “Type 2” portion 1567 at least partly in response to descriptive data 1565). This can occur, for example, in embodiments in which configuration circuitry 1570 performs operation 860 (alone or jointly with interface module 1550, e.g.). The descriptive data relating to clinical data 1563 can, for example, include an owner, an input device identifier, a sample or summary of clinical data 1563, or the like. A relation between such descriptive information and clinical data 1563 can be obtained, for example, by including the information within the data, by a categorization or known arrangement of the data, by applying data format evaluation or other criteria, or the like. In some embodiments, a preliminary indication of compatibility can be inferred in response to an error message, a type list, an allowable range of values, or the like. Alternatively or additionally, such criteria can be derived by trying to process the data portions with “A” type app(s) 1591, and by indicating an apparent compatibility in response to a suitable interval without an error report or the like, or to some other indication of successful processing.

Operation 2664 describes associating the pointer for the clinical analysis module with a conditional invocation in the one or more applications (e.g. association logic 1575 associating pointer 1542 with a jump operation that is only performed if a variable is TRUE, in the one or more “A” type apps 1591). In some embodiments, of course, the invocation can reside in a non-branching code block that is only performed if a specific condition exists (e.g. within a local program branch of “A” type apps 1591, performed if a computed result is equal to zero). Alternatively or additionally, the invocation can request processing in a remote task or instance of analysis modules 1400, for example, or queue or otherwise trigger some instance of such a module.

Operation 2667 describes receiving user input identifying the one or more applications and at least a portion of the clinical analysis module (e.g. input device 1573 receiving typed data, menu selections, voice data, or the like specifying an app type or other descriptors, a pathological or other physiological term, a data type, a task attribute, or other information distinguishing the selected tasks, applications or code functionality from others). Those skilled in the art will recognize a variety of ways of including or excluding “A” type app(s) 1591, for example, from the set to be configured. The user input can be received from user interface 715 via network interface 730, in some embodiments, such as those in which primary system 700 implements control module 1540 as described above. Alternatively or additionally, the user input can include pointer 1543 or other indications of any clinical analysis module(s) to be enabled.

With reference now to FIG. 27, there are shown several variants of the flow 1000 of FIG. 10. Operation 1030—receiving an indication of an anomalous device-interactive performance of a specific individual—may include one or more of the following operations: 2732, 2733, 2738 or 2739. Operation 1040—selecting one or more diagnostic instructions at least partly based on the anomalous device-interactive performance of the specific individual—may include one or more of the following operations: 2741, 2742, 2744, 2745, 2748, or 2749.

Operation 2732 describes applying one or more premises relating to a diagnostic group to performance data relating to a member of the diagnostic group (e.g. normative logic 1958 applying a pulse rate range for 42-year-old women to evaluate whether a specific 42-year-old woman has a normal pulse rate for her age). The diagnostic group can be bounded by age range, gender or other genetic attribute, symptom or other medical status, activity type, affiliation, interface type, linguistic or cultural norms, geography, or the like.

Operation 2733 describes detecting one or more non-anomalous performances (e.g. anomaly detection logic 1464 or the like indicating data 1484 as “normal” or otherwise non-anomalous). In various embodiments, such logic can implement one or more of analysis module(s) 1400 of FIG. 14, for example. In some embodiments, a portion of the non-anomalous data can be retained by sample selection logic 1457, for example, as samples or other indications representative of a normal condition of the individual(s). Alternatively or additionally, sample selection logic 1457 can preferentially select anomaly-indicative samples in various ways in light of teachings herein. In this way, for example, normal fluctuations can be objectively and economically distinguished from apparent transitional events or trends (suddenly or gradually increasing an addictive behavior by 50% or more, for example, such as smoking or online game play).

Operation 2738 describes performing timing analysis at least on the indication of the anomalous device-interactive performance (e.g. interval detection logic 1469 or the like determining whether a reaction time or the like was within a normal range). This can occur, for example, in embodiments in which device 900 or intake module 1940 includes one or more instances of analysis module(s) 1400, in which intake module 1940 performs operation 1030, and in which analysis module 1960 or other portions of system 1900 perform other operations of flow 1000. Determining that a timing aspect of the device-interactive performance was abnormal can, in some embodiments, trigger an inference that the performance was anomalous. In some variants, a priori knowledge of the specific individual (e.g. age, past performance, or the like) can enhance the accuracy of such normalcy thresholds, as described herein.

Operation 2739 describes receiving leisure activity performance data relating to the specific individual (e.g. port 941 receiving data relating to individual 930 or some other individual performing some device-interactive leisure activity). The leisure activity can include a conversation via a telephonic device or keyboard, playing an instrument or computer game, going for a drive, or some other leisure activity performed by interacting with a system like that of device 900. Such data can be useful in many ways as described herein, such as by a processor-based system that can record it or otherwise detect it in some fashion.

Operation 2741 describes generating a message indicating the one or more diagnostic instructions (e.g. message generator 1964 signaling a clinician to administer a patient questionnaire, a blood test, or some other diagnostic instrument that depends at least partly on anomaly indications 956). Alternatively or additionally, the message can direct one or more analysis modules to be performed upon performance data 1952 or the like to facilitate a diagnosis, in light of teachings herein.

Operation 2742 describes generating an evaluation by performing the one or more diagnostic instructions (e.g. evaluation module 1969 generating a computed variance, a sample of data selected as representative, a phrase, a percentile, or the like to describe qualitative or quantitative attributes of the anomaly or other aspect of the performance). In some embodiments, for example, the evaluation can include a text value of “+,” an error message, “irregular,” a form paragraph, or the like.

Operation 2744 describes selecting the one or more diagnostic instructions partly based on a frequency or a duration (e.g. branch logic 967 selecting instructions 964 in lieu of instructions 965 because a computed result indicating that a computed duration was negative). This can occur, for example, when the negative result indicates that one event happened before another (e.g. a deadline expiring before the specific individual responds). A kind of positive result can likewise occur, such as when the individual succeeds with an unusually high frequency. A diagnostician can fairly infer a cognitive or sensory improvement from a suddenly higher frequency of success, for example, which can be helpful for evaluating whether a new regimen was helpful. In some embodiments, of course, branch logic 967 can likewise be configured to decide among access object(s) 968 or instructions 964 based on durations, frequencies, or other time-related computations. Those skilled in the art will recognize many such embodiments in light of teachings herein.

Operation 2745 describes receiving input data signaling a selection of the one or more diagnostic instructions (e.g. one or more instances of interfaces 940 detecting a user action indicating a selection validation indicator or selection indicating one or more analysis module(s) 1400). In some embodiments, for example, such a selection can designate a menu option indicating an instruction series. Alternatively or additionally, the selection can be performed before operation 1030 is complete and so that the selection controls how later-received anomaly indications will be treated.

Operation 2748 describes selecting the one or more diagnostic instructions in response to a symptom (e.g. pathology-specific logic 1463 selecting kinesthetic analysis logic 1465 or the like in response to images and auditory data indicating that a patient may be suffering from increasing symptoms of a joint disorder). The symptoms may include a popping sound or facial or verbal expression of pain in conjunction with a joint motion, in some embodiments. Others of analysis module(s) 1400 may likewise be selected in appropriate circumstances, such as by selecting cardiological analysis logic 1462 in response to an indication of potential heart trouble. Alternatively or additionally, a user interface as described herein can show a physician a list of available analysis modules relating to a symptom of pain evident to the physician in a video clip of a patient.

Operation 2749 describes changing one or more anomalous performance detection criteria in response to an output from the one or more diagnostic instructions (e.g. some instance of anomaly detection logic 1464 incrementally expanding a normal range defined in data 2036 in response to an indication of too many false positives in anomaly indication(s) 956). Conversely a set of normal values defined in data 2036 can be incrementally reduced in response to an indication of too many false negatives, in some embodiments, so that configuration circuitry as described herein can cause anomaly detection logic 1464 to draw closer to an optimal sensitivity adaptively.

With reference now to FIG. 28, there are shown several variants of the flow 1000 of FIG. 10 or 27. Operation 1030—receiving an indication of an anomalous device-interactive performance of a specific individual—may include one or more of the following operations: 2831, 2833, 2834, 2836 or 2838. Alternatively or additionally flow 1000 may likewise include one or more of operations 2861, 2864, 2866, 2868, or 2869—each depicted after operation 1040 but optionally performed concurrently with it, or in other orders.

Operation 2831 describes detecting a performance anomaly at least by comparing a scalar performance indicator with a threshold (e.g. comparator 1951 indicating whether performance data 1952 indicates an apparent anomaly in response to measuring a recent success rate of 60% in comparison to a historical success range of 72% to 84%). Likewise a performance anomaly can be recognized in response to an average power level increasing significantly (by at least about a decibel in some contexts, e.g.). A change of these types can optionally be detected by a applying a minimum or maximum threshold to a scalar performance indicator such as a ratio, an absolute percentage change, a computed variance, a number of metric units, or the like. Those skilled in the art will recognize a wide variety of scalar thresholds, and groups thereof, suitable for distinguishing anomalies from insignificant fluctuation in light of these teachings.

Operation 2833 describes detecting an anomaly after the anomalous device-interactive performance of the specific individual ends (e.g. scanning logic 1956 indicating which members of a population of test subjects exhibit a recognizable performance anomaly). The anomaly may be initially recognized as matching one or more performance pattern(s) 1957 essentially in the same manner that human beings can be observed to recognize any of a variety of patterns, often unconsciously. Fatigue-indicative patterns, for example, can include yawning, sluggishness, slow reactions, diminished functional performance, or the like. Many such patterns can readily be recognized readily available image recognition techniques and applied in light of teachings herein.

In some embodiments, scanning logic 1956 can recognize such patterns in data, such as by applying image analysis logic 1453 to data 2034 from an earlier device-interactive performance of the specific individual(s). In some embodiments, at least some raw data from many individuals is archived for later analysis by subsequent versions of one or more instances of analysis modules 1960.

Operation 2834 describes detecting an anomaly during the anomalous device-interactive performance of the specific individual (e.g. monitoring logic 1953 detecting a sign of apparently severe distress in a user's interaction with or through a handheld device during the interaction or perhaps at least within a diagnostically appropriate period of the interaction). This can occur, for example, in embodiments in which intake module 1940 performs operation 1030. Depending on one or more pathologies of the individual, such periods may comprise a few minutes, a few hours, a few days, a few months, or even longer. In some embodiments herein, such interactions can include a conversation, a transaction or session, or the like. Such signs (sobbing, screaming, fleeing, or the like) can be recognized from audio, image, or motion data in the device in some embodiments described herein, or from other sensors nearby.

Operation 2836 describes applying at least auditory processing of the indication of the anomalous device-interactive performance of the specific individual (e.g. detection logic 1954 including auditory analysis logic 1451 or the like configured for distilling at least some category or other indication of a telephonic conversation or other device-interactive performance as described herein). In some embodiments, the auditory processing can include a filter that preferentially retains vocal-range frequency signals, for example. Alternatively or additionally, a filter can be used for removing time segments of audio data with no recognizable sounds. Those skilled in the art will recognize in light of these teachings that such techniques tend to enhance a performance of the retained data (such as can be indicated by a signal-to-noise ratio, as a speech density in words or recognizable inflections per unit of storage or display, or the like).

Operation 2838 describes configuring a device according to an attribute of the specific individual (e.g. storage manager 1942 loading media 1944 with one or more analysis module(s) 1400 selected for use for the specific individual, or one or more instances of values 1945 therefrom). In some embodiments, the device configuration can be customized according to the individual's gender, age, symptom(s), or other medical history. Such analysis module(s) 1400 can be selected programmatically by a pathology or the like, for example, or according to authorizations, default values, or other information such as may be provided by physician or other authorized caregiver.

Operation 2861 describes obtaining information confirming an identity of the specific individual after the anomalous device-interactive performance of the specific individual (e.g. subject identification logic 1468 recording photographic or other biometric data, requesting a password, or otherwise authenticating an identity of one or more of users 130, 335, party 1102, or specific individual 930). Such authentications or the like can occur, for example, in any of several variants of flows 200, 400, 1000, 1200 described herein as an appropriate response to receiving data or taking other actions described herein, generally to affirm or otherwise assure the appropriateness of responsive actions.

Operation 2864 describes performing a transmission of or a reception of an instruction sequence including at least the one or more diagnostic instructions (e.g. update module 1962 transmitting or receiving code that includes a sequence of diagnostic instructions). This can implement a local or remote update of analysis module(s) 1400 as described herein, or of applications containing analysis module(s) 1400, in response to the anomalous device-interactive performance(s) of the specific individual.

Operation 2866 describes executing the one or more diagnostic instructions (e.g. processor 1927 executing code selected at least partly based on the device-interactive performance). In some embodiments, the type or timing of detected anomalies can at least partly control when or how the diagnostic instruction(s) are executed. In some embodiments, for example, an anomaly of interest primarily for research purposes can trigger a lower priority execution of the pertinent code than that of a crisis-indicative anomaly.

Operation 2868 describes causing the one or more diagnostic instructions to generate a diagnosis (e.g. processor 1928 activating one or more analysis modules 1400 resulting in at least one diagnosis of one or more attributes of the specific individual). In an embodiment in which one or more analysis modules 1400 includes an ability to recognize the indication of the anomalous performance as a false positive, for example, the diagnosis be null, “normal” or the like. This can occur, for example, in embodiments in which the one or more analysis modules 1400 can infer from a fuller analysis that the false positive apparently resulted from a data glitch or other cause unrelated to the specific individual.

Operation 2869 describes requesting the one or more diagnostic instructions (e.g. request logic 1967 requesting one or more instructions selected in operation 1040 from a remote device, not shown). Alternatively or additionally, a library or other collection that includes such instruction(s) can be requested before or during operation 1040, and the outcome of the selection can be used for deciding which such instruction(s) to execute. In some embodiments, the instruction(s) selected can be obtained and then implemented in applications as described herein.

With reference now to FIG. 29, there are shown several variants of the flow 1200 of FIG. 12. Operation 1250—signaling a decision whether to notify a first party at least by applying a first screen to one or more health-status-indicative updates relating to a second party—may include one or more of the following operations: 2951, 2953, 2957, or 2958. Operation 1280—signaling a decision whether to notify a third party at least by applying a second screen to the one or more health-status-indicative updates relating to the second party—may include one or more of the following operations: 2982, 2983, 2986, or 2988.

Operation 2951 describes determining whether the one or more health-status-indicative updates indicate a new symptom (e.g. sensor 1410 detecting that a symptom-indicative parameter has changed by a magnitude large enough to support an inference that the “second” party has a new symptom). This can occur, for example, in embodiments in which one or more instances of sensors 1410, 1421 or other logic 1451-1469 can perform operation 1250 and in which one or more instances of distribution circuitry 1440 or transmitters 1430 can perform operation 1280.

Those skilled in the art will recognize that in some contexts any patient status change will constitute a “new symptom” inference: yes/no determinations of whether the second party/user has a pulse, can respond, or the like, for example. In some embodiments, sensor 1410 can implement one or more criteria 1414 for making such determinations: by computing a difference between or otherwise comparing a past-symptom or range vector 1415 with a recent-status vector 1416, for example. If the “first” party's notification profile includes a reference to a just-manifested symptom (or to all new symptoms), distribution circuitry 1440 can implement such profiles in one or more distribution modes 1444.

Operation 2953 describes determining whether the one or more health-status-indicative updates indicate a crisis (e.g. sensor 1421 applying one or more physician- or other expert-defined thresholds or other crisis-indicative criteria 1422 to determine whether or which parties 1101-1104 should be notified). The “first” party may be a physician or ambulance service, for example, for an audible or other significant indication that a heart patient or other at-risk “second” party may be suffering a heart attack, a seizure, a condition causing tremors or other observable symptoms, an auto wreck, complications from a surgery, or the like. In some distribution modes 1441-1449 of “third” parties, the inclusion or responses of one or more “first” parties may contraindicate the decision whether to notify a “third” party.

Operation 2957 describes associating the first screen with a distribution list including at least the first party (e.g. one or more instances of linkages 1311-1319 associating one or more data type(s) 1331-1339 with corresponding access information 1351-1359 authorizing notification to or other access by the “first” party). This can occur, for example, in embodiments in which the first party includes one or more of parties 1101, 1102; in which invocation module 1130 has access to or otherwise implements one or more linkages 1311-1319 of association circuitry 1300 or other association logic 1576, in which at least some decision circuitry 1141, 1142 performs operation 1250, and in which at least some other decision circuitry 1142, 1143 or external circuitry 1190 performs operation 1280.

Operation 2958 describes determining whether the one or more health-status-indicative updates indicate a health deterioration (e.g. sensor 1427 applying one or more first-party-defined thresholds or other criteria 1428 to some or all of data 1491-1499 to determine whether a potential health deterioration is apparent therefrom). Sensor 1427 can be configured, for example, by party 1105 defining screen A 1151 so as to be notified whenever data 1493 indicates a body temperature (of party 1101, e.g.) above 39° Celsius or a pulse rate of 140 BPM, or further increases therefrom. Substantially all such notices can, for some pathologies, signal a health status deterioration. Many types of health status deteriorations are detectable in embodiments herein, such as can manifest in a pathology-indicative quantity or the like becoming abnormal or more abnormal. In some variants, for example, an apparent health status decline of one or more users 130, 335 is detected at least partly based on one or more worsening or other worse-than-nominal performances in a surreptitiously observed or occupational or leisure interaction as described herein. Those skilled in the art can recognize such quantities as they reflect deteriorations in the “second” party or user in relation to his/her normal range or that of one or more others. In some cases a hypothesis relating to the deterioration (e.g. drunkenness, exhaustion, overdose, injury, illness, or the like) can be obtained from context information (conversation, e.g.) or directly (from a caregiver who identifies a pathology, e.g.).

Operation 2982 describes notifying the first party and the third party of a result of the second screen (e.g. distribution circuitry 1440 applying mode 1449 to notify two or more caregivers or the like). This can be desirable, for example, in contexts in which the second screen detects an unusually significant event: a stroke, arrhythmia, an auto accident, a physical attack or the like apparent bearing upon a health status of at least the “second” party.

Operation 2983 describes transmitting to the third party the one or more health-status-indicative updates including at least a selected sample of raw data (e.g. invoker 1432 causing party 1103 to receive remotely archived or other user-entered data, video or audio segments, or other such raw data 2039 significantly indicative of a health status evaluation of party 1102 relying on the raw data). This can occur, for example, in embodiments in which some or all analysis module(s) 1400 or data-containing media 2030 available to primary system 1130 are distributed across more than one site. Such raw data can ordinarily be of immediate interest to the “third” party, for example, to corroborate or perhaps at least elaborate upon an evaluation or other “second” screen triggering the notification decision.

Operation 2986 describes notifying the second party and the third party of a result of the second screen (e.g. distribution circuitry 1440 applying mode 1447). A distribution list, party inclusion or selection criteria, or like can be used as mode 1447 by which distribution circuitry 1440 notifies at least the “second” and “third” parties. In some embodiments a patient (as the “second” party, e.g.) wishes to notify a third party automatically in response to a debilitating event (as the “second” screen, e.g.) so that the patient's chosen third party will receive prompt notice. Operation 2986 can cause such a patient to receive similar notice (if conscious), serving as a roughly contemporaneous indication that the chosen third party is being notified.

Operation 2988 describes archiving the decision whether to notify the third party (e.g. aggregator 1770 implementing filter 1771 by recording at least a partial list of who was or should be notified). This can occur, for example, in embodiments in which configuration module 1408 provides or adapts filter 1771 according to selections received from one or more users (as the “second” party) described herein. The archiving can optionally occur before or in some other temporal relation to such notification, in some embodiments. Alternatively or additionally, some variants of operation 2988 can be performed in relation to a “first” party (or some other user) and combined with one or more other user-related operations described herein.

With reference now to FIG. 30, there are shown several variants of the flow 400 of FIG. 4. Operation 410—receiving health-status-indicative data surreptitiously captured from an interaction between a device and a user—may include one or more of the following operations: 3012, 3015, or 3019. Operation 440—applying one or more data extraction criteria to the health-status-indicative data surreptitiously captured from the interaction between the device and the user—may include one or more of the following operations: 3043, 3044, 3046, 3048, or 3049.

Operation 3012 describes receiving leisure activity performance data of the health-status-indicative data (e.g. port 1754 receiving at least user game moves, scores, screen captures, social interactions, browsing selections or the like of which some might reasonably be expected to indicate any substantial emotional or personality shifts, or similar health status data). This can occur, for example, in embodiments in which one or more instances of input modules 371 or interfaces 1610, 1720 perform operation 410, in which one or more instances of distillation modules 1660, 1790 or extraction modules 333 perform operation 440, or in which system 1600 is implemented as a portable device or other stand-alone system, or within an embodiment of system 1700.

Operation 3015 describes receiving the health-status-indicative data substantially contemporaneously with the interaction (e.g. recorder 1780 surreptitiously capturing image or audio data from an interaction between an infant and a toy). In some variants, recorder can include filter 1781 operable for pausing the recorder or otherwise excluding or marking less-desirable data: data that is not surreptitious or is unrelated to the interaction between the device and the user. Alternatively or additionally, recorder can include filter 1782 operable for excluding or identifying (at least some) data that is not indicative of health status, such as by activating the recording only in response to an expression of a symptom.

Operation 3019 describes configuring at least the device to capture the health-status-indicative data surreptitiously (e.g. configuration logic 1718 adapting one or more instances of interfaces 1610, 1720 of devices 330 so that the user will not be conscious of being monitored). In some variants in which user 335 consents to being monitored, for example, configuration logic 1718 causes at most a subtle reminder (or no reminder) to be presented to the user real time, for most or all of the capture period(s).

Operation 3043 describes selecting extraction logic in response to input from an interface of the device (e.g. selection logic 1691 selecting one or more extraction modes 1695-1697 or the like in response to input 1612 from interface 1610). Extraction mode 1695 can include a clip selection mode or the like, for example, in response to a performance anomaly or like indication that some video or audio data of input 1612 may be more likely to be relevant than a remainder of the video or audio data. Alternatively or additionally, in a wireless implementation or other context in which a distribution bottleneck can occur, control logic can instead implement mode 1696, by which data types and times are provided at least initially in lieu of video or audio footage.

Operation 3044 describes applying one or more extraction modes selected in response to data-type-indicative information (e.g. extraction logic 1778 applying mode 1697 selected at least partly in response to “JPG,” “language,” “MP3” or other data type or format category 1632 from category selector 1630). Such modes can optionally be selected in response to one or more predetermined attributes of the user such as at association with some data 2031-2039 obtained earlier as described herein (optionally via data processor 1650 or the like).

Operation 3046 describes selecting the one or more data extraction criteria at least partly based on information relating to the user (e.g. selection logic 1692 selecting translator 1671 or the like for use in or with filter 1674 or other extraction logic, in response to a priori or detected language data 2037 indicating that the user speaks or writes in Lithuanian). This can sometimes matter in a context in which such criteria can facilitate further analysis as described herein (which may not otherwise function in Lithuanian, for example). Other such helpful user-related information can include one or more instances of health status (age, gender, genetic background, medical history, current medications, or the like), location, software or device usage habits, sleep habits, or the like.

Operation 3048 describes selecting the one or more data extraction criteria at least partly based on a menu selection (e.g. selection logic 1694 selecting at least criterion 1698 according to an option selection received as input 1612). This can occur, for example, in embodiments in which system 1600 includes one or more instances of interfaces 1720 or recorders 1780, in which at least interface 1720 performs operation 410, and in which one or more instances of control logic 1690 or distillation modules 1660 perform operation 440.

Operation 3049 describes deciding whether to signal a party partly based on the health-status-indicative data surreptitiously captured from the interaction between the device and the user (e.g. selection logic 1693 selecting the “first” party of FIG. 12 in response to an indication that this party should be notified of surreptitiously captured data). In the even that even surreptitiously captured data objectively indicates that the user is becoming more manic, for example, providing this indication directly to the user's doctor (by e-mail or automatic voice message, e.g.) may help the doctor be more prompt in adjusting the user's dosage (of lithium or the like, e.g.). The decision whether to signal the party can also depend on one or more of a time of day, the party's location or other status information, the user's location or other status information, medical or other historical information, other data relating to the interaction or to the device, or the like.

The foregoing detailed description has set forth various embodiments of the devices and/or processes via the use of block diagrams, flowcharts, and/or examples. Insofar as such block diagrams, flowcharts, and/or examples contain one or more functions and/or operations, it will be understood by those within the art that each function and/or operation within such block diagrams, flowcharts, or examples can be implemented, individually and/or collectively, by a wide range of hardware, software, firmware, or virtually any combination thereof. In one embodiment, several portions of the subject matter described herein may be implemented via Application Specific Integrated Circuits (ASICs), Field Programmable Gate Arrays (FPGAs), digital signal processors (DSPs), or other integrated formats. However, those skilled in the art will recognize that some aspects of the embodiments disclosed herein, in whole or in part, can be equivalently implemented in integrated circuits, as one or more computer programs running on one or more computers (e.g., as one or more programs running on one or more computer systems), as one or more programs running on one or more processors (e.g., as one or more programs running on one or more microprocessors), as firmware, or as virtually any combination thereof, and that designing the circuitry and/or writing the code for the software and or firmware would be well within the skill of one of skill in the art in light of this disclosure. In addition, those skilled in the art will appreciate that the mechanisms of the subject matter described herein are capable of being distributed as a program product in a variety of forms, and that an illustrative embodiment of the subject matter described herein applies regardless of the particular type of signal bearing medium used to actually carry out the distribution. Examples of a signal bearing medium include, but are not limited to, the following: a recordable type medium such as a floppy disk, a hard disk drive, a Compact Disc (CD), a Digital Video Disk (DVD), a digital tape, a computer memory, etc.; and a transmission type medium such as a digital and/or an analog communication medium (e.g., a fiber optic cable, a waveguide, a wired communications link, a wireless communication link, etc.).

In a general sense, those skilled in the art will recognize that the various embodiments described herein can be implemented, individually and/or collectively, by various types of electro-mechanical systems having a wide range of electrical components such as hardware, software, firmware, or virtually any combination thereof; and a wide range of components that may impart mechanical force or motion such as rigid bodies, spring or torsional bodies, hydraulics, and electro-magnetically actuated devices, or virtually any combination thereof. Consequently, as used herein “electro-mechanical system” includes, but is not limited to, electrical circuitry operably coupled with a transducer (e.g., an actuator, a motor, a piezoelectric crystal, etc.), electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), electrical circuitry forming a communications device (e.g., a modem, communications switch, or optical-electrical equipment), and any non-electrical analog thereto, such as optical or other analogs. Those skilled in the art will also appreciate that examples of electro-mechanical systems include but are not limited to a variety of consumer electronics systems, as well as other systems such as motorized transport systems, factory automation systems, security systems, and communication/computing systems. Those skilled in the art will recognize that electro-mechanical as used herein is not necessarily limited to a system that has both electrical and mechanical actuation except as context may dictate otherwise.

In a general sense, those skilled in the art will recognize that the various aspects described herein which can be implemented, individually and/or collectively, by a wide range of hardware, software, firmware, or any combination thereof can be viewed as being composed of various types of “electrical circuitry.” Consequently, as used herein “electrical circuitry” includes, but is not limited to, electrical circuitry having at least one discrete electrical circuit, electrical circuitry having at least one integrated circuit, electrical circuitry having at least one application specific integrated circuit, electrical circuitry forming a general purpose computing device configured by a computer program (e.g., a general purpose computer configured by a computer program which at least partially carries out processes and/or devices described herein, or a microprocessor configured by a computer program which at least partially carries out processes and/or devices described herein), electrical circuitry forming a memory device (e.g., forms of random access memory), and/or electrical circuitry forming a communications device (e.g., a modem, communications switch, or optical-electrical equipment). Those having skill in the art will recognize that the subject matter described herein may be implemented in an analog or digital fashion or some combination thereof.

Those skilled in the art will recognize that it is common within the art to describe devices and/or processes in the fashion set forth herein, and thereafter use engineering practices to integrate such described devices and/or processes into image processing systems. That is, at least a portion of the devices and/or processes described herein can be integrated into an image processing system via a reasonable amount of experimentation. Those having skill in the art will recognize that a typical image processing system generally includes one or more of a system unit housing, a video display device, a memory such as volatile and non-volatile memory, processors such as microprocessors and digital signal processors, computational entities such as operating systems, drivers, and applications programs, one or more interaction devices, such as a touch pad or screen, control systems including feedback loops and control motors (e.g., feedback for sensing lens position and/or velocity; control motors for moving/distorting lenses to give desired focuses. A typical image processing system may be implemented utilizing any suitable commercially available components, such as those typically found in digital still systems and/or digital motion systems.

Those skilled in the art will recognize that it is common within the art to describe devices and/or processes in the fashion set forth herein, and thereafter use engineering practices to integrate such described devices and/or processes into data processing systems. That is, at least a portion of the devices and/or processes described herein can be integrated into a data processing system via a reasonable amount of experimentation. Those having skill in the art will recognize that a typical data processing system generally includes one or more of a system unit housing, a video display device, a memory such as volatile and non-volatile memory, processors such as microprocessors and digital signal processors, computational entities such as operating systems, drivers, graphical user interfaces, and applications programs, one or more interaction devices, such as a touch pad or screen, and/or control systems including feedback loops and control motors (e.g., feedback for sensing position and/or velocity; control motors for moving and/or adjusting components and/or quantities). A typical data processing system may be implemented utilizing any suitable commercially available components, such as those typically found in data computing/communication and/or network computing/communication systems.

Those skilled in the art will recognize that it is common within the art to implement devices and/or processes and/or systems in the fashion(s) set forth herein, and thereafter use engineering and/or business practices to integrate such implemented devices and/or processes and/or systems into more comprehensive devices and/or processes and/or systems. That is, at least a portion of the devices and/or processes and/or systems described herein can be integrated into other devices and/or processes and/or systems via a reasonable amount of experimentation. Those having skill in the art will recognize that examples of such other devices and/or processes and/or systems might include—as appropriate to context and application—all or part of devices and/or processes and/or systems of (a) an air conveyance (e.g., an airplane, rocket, hovercraft, helicopter, etc.), (b) a ground conveyance (e.g., a car, truck, locomotive, tank, armored personnel carrier, etc.), (c) a building (e.g., a home, warehouse, office, etc.), (d) an appliance (e.g., a refrigerator, a washing machine, a dryer, etc.), (e) a communications system (e.g., a networked system, a telephone system, a Voice over IP system, etc.), (f) a business entity (e.g., an Internet Service Provider (ISP) entity such as Comcast Cable, Quest, Southwestern Bell, etc), or (g) a wired/wireless services entity such as Sprint, Cingular, Nextel, etc.), etc.

All of the above-mentioned U.S. patents, U.S. patent application publications, U.S. patent applications, foreign patents, foreign patent applications and non-patent publications referred to in this specification and/or listed in any Application Data Sheet, are incorporated herein by reference, to the extent not inconsistent herewith.

One skilled in the art will recognize that the herein described components (e.g., steps), devices, and objects and the discussion accompanying them are used as examples for the sake of conceptual clarity and that various configuration modifications are within the skill of those in the art. Consequently, as used herein, the specific exemplars set forth and the accompanying discussion are intended to be representative of their more general classes. In general, use of any specific exemplar herein is also intended to be representative of its class, and the non-inclusion of such specific components (e.g., steps), devices, and objects herein should not be taken as indicating that limitation is desired.

Although users 130, 335, 1851 are typically shown and described herein each as a single illustrated figure, those skilled in the art will appreciate that such users may be representative of a human user, a robotic user (e.g., computational entity), and/or substantially any combination thereof (e.g., a user may be assisted by one or more robotic agents). In addition, each such user, as set forth herein, although shown as a single entity may in fact be composed of two or more entities. Those skilled in the art will appreciate that, in general, the same may be said of “sender” and/or other entity-oriented terms as such terms are used herein.

With respect to the use of substantially any plural and/or singular terms herein, those having skill in the art can translate from the plural to the singular and/or from the singular to the plural as is appropriate to the context and/or application. The various singular/plural permutations are not expressly set forth herein for sake of clarity.

The herein described subject matter sometimes illustrates different components contained within, or connected with, different other components. It is to be understood that such depicted architectures are merely exemplary, and that in fact many other architectures can be implemented which achieve the same functionality. In a conceptual sense, any arrangement of components to achieve the same functionality is effectively “associated” such that the desired functionality is achieved. Hence, any two components herein combined to achieve a particular functionality can be seen as “associated with” each other such that the desired functionality is achieved, irrespective of architectures or intermedial components. Likewise, any two components so associated can also be viewed as being “operably connected”, or “operably coupled”, to each other to achieve the desired functionality, and any two components capable of being so associated can also be viewed as being “operably couplable”, to each other to achieve the desired functionality. Specific examples of operably couplable include but are not limited to physically mateable and/or physically interacting components and/or wirelessly interactable and/or wirelessly interacting components and/or logically interacting and/or logically interactable components.

While particular aspects of the present subject matter described herein have been shown and described, it will be apparent to those skilled in the art that, based upon the teachings herein, changes and modifications may be made without departing from the subject matter described herein and its broader aspects and, therefore, the appended claims are to encompass within their scope all such changes and modifications as are within the true spirit and scope of the subject matter described herein. Furthermore, it is to be understood that the invention is defined by the appended claims. It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to inventions containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

With respect to the appended claims, those skilled in the art will appreciate that recited operations therein may generally be performed in any order. Examples of such alternate orderings may include overlapping, interleaved, interrupted, reordered, incremental, preparatory, supplemental, simultaneous, reverse, or other variant orderings, unless context dictates otherwise. With respect to context, even terms like “responsive to,” “related to,” or other past-tense adjectives are generally not intended to exclude such variants, unless context dictates otherwise.

While various aspects and embodiments have been disclosed herein, other aspects and embodiments will be apparent to those skilled in the art. The various aspects and embodiments disclosed herein are for purposes of illustration and are not intended to be limiting, with the true scope and spirit being indicated by the following claims.

1-27. (canceled)

28. A method comprising:
obtaining data from occupational or leisure intercommunication at least between a device and a user; and
signaling a data distillation indicating a health status change of the user at least partly based on the data from the occupational or leisure intercommunication.

29. (canceled)

30. The method of claim 28 in which obtaining data from occupational or leisure intercommunication at least between a device and a user comprises:
receiving an identifier of the user with the data from the occupational or leisure intercommunication.

31-33. (canceled)

34. The method of claim 28 in which signaling a data distillation indicating a health status change of the user at least partly based on the data from the occupational or leisure intercommunication comprises:
causing a module to process at least a natural language expression from the data.

35. (canceled)

36. The method of claim 28 in which signaling a data distillation indicating a health status change of the user at least partly based on the data from the occupational or leisure intercommunication comprises:
applying one or more normalcy criteria to at least a portion of the data from the occupational or leisure intercommunication.

37. (canceled)

38. The method of claim 28 in which signaling a data distillation indicating a health status change of the user at least partly based on the data from the occupational or leisure intercommunication comprises:
generating an expression indicating a composition of matter at least partly based on the data.

39-43. (canceled)

44. The method of claim 28 in which signaling a data distillation indicating a health status change of the user at least partly based on the data from the occupational or leisure intercommunication comprises:
removing a portion of the data from the occupational or leisure intercommunication.

45-47. (canceled)

48. The method of claim 28 in which obtaining data from occupational or leisure intercommunication at least between a device and a user comprises:
configuring a data acquisition mode of the device in response to information about the user.

49. (canceled)

50. The method of claim 28 in which obtaining data from occupational or leisure intercommunication at least between a device and a user comprises:
obtaining auditory information in the data from the occupational or leisure intercommunication.

51-54. (canceled)

55. A system comprising:
means for obtaining data from occupational or leisure intercommunication at least between a device and a user; and
means for signaling a data distillation indicating a health status change of the user at least partly based on the data from the occupational or leisure intercommunication.

56. The system of claim 55 in which the means for obtaining data from occupational or leisure intercommunication at least between a device and a user comprises:
means for receiving the data from the occupational or leisure intercommunication remotely.

57. The system of claim 55 in which the means for obtaining data from occupational or leisure intercommunication at least between a device and a user comprises:
means for receiving an identifier of the user with the data from the occupational or leisure intercommunication.

58. The system of claim 55 in which the means for obtaining data from occupational or leisure intercommunication at least between a device and a user comprises:
means for receiving the data at the device.

59. (canceled)

60. The system of claim 55 in which the means for obtaining data from occupational or leisure intercommunication at least between a device and a user comprises:
means for receiving data from other interaction between the user and the device.

61. The system of claim 55 in which the means for signaling a data distillation indicating a health status change of the user at least partly based on the data from the occupational or leisure intercommunication comprises:
means for causing a module to process at least a natural language expression from the data.

62. The system of claim 55 in which the means for signaling a data distillation indicating a health status change of the user at least partly based on the data from the occupational or leisure intercommunication comprises:
means for causing a module to process at least some user input from the data.

63. The system of claim 55 in which the means for signaling a data distillation indicating a health status change of the user at least partly based on the data from the occupational or leisure intercommunication comprises:
means for applying one or more normalcy criteria to at least a portion of the data from the occupational or leisure intercommunication.

64. The system of claim 55 in which the means for signaling a data distillation indicating a health status change of the user at least partly based on the data from the occupational or leisure intercommunication comprises:
means for using at least a portion of the data that originated from a leisure activity.

65. The system of claim 55 in which the means for signaling a data distillation indicating a health status change of the user at least partly based on the data from the occupational or leisure intercommunication comprises:
means for generating an expression indicating a composition of matter at least partly based on the data.

66. The system of claim 55 in which the means for obtaining data from occupational or leisure intercommunication at least between a device and a user comprises:
means for retrieving the data from a capture archive.

67. The system of claim 55 in which the means for obtaining data from occupational or leisure intercommunication at least between a device and a user comprises:
means for obtaining the data from one or more messages sent from the device.

68. The system of claim 55 in which the means for obtaining data from occupational or leisure intercommunication at least between a device and a user comprises:
means for receiving the data from one or more stationary sensors during the occupational or leisure intercommunication.

69. The system of claim 55 in which the means for signaling a data distillation indicating a health status change of the user at least partly based on the data from the occupational or leisure intercommunication comprises:
means for requesting an extraction of a portion of the data indicating when an ability of the user apparently changed.

70. (canceled)

71. The system of claim 55 in which the means for signaling a data distillation indicating a health status change of the user at least partly based on the data from the occupational or leisure intercommunication comprises:
means for removing a portion of the data from the occupational or leisure intercommunication.

72. The system of claim 55 in which the means for signaling a data distillation indicating a health status change of the user at least partly based on the data from the occupational or leisure intercommunication comprises:
means for distilling the data from the occupational or leisure intercommunication from other data.

73-74. (canceled)

75. The system of claim 55 in which the means for obtaining data from occupational or leisure intercommunication at least between a device and a user comprises:
means for configuring a data acquisition mode of the device in response to information about the user.

76. The system of claim 55 in which the means for obtaining data from occupational or leisure intercommunication at least between a device and a user comprises:
means for obtaining optical information in the data from the occupational or leisure intercommunication.

77. The system of claim 55 in which the means for obtaining data from occupational or leisure intercommunication at least between a device and a user comprises:
means for obtaining auditory information in the data from the occupational or leisure intercommunication.

78. The system of claim 55 in which the means for signaling a data distillation indicating a health status change of the user at least partly based on the data from the occupational or leisure intercommunication comprises:
means for receiving an event type indication and a date indication in the data from the occupational or leisure intercommunication; and
means for signaling the event type indication and the date indication in the data distillation.

79. The system of claim 55 in which the means for signaling a data distillation indicating a health status change of the user at least partly based on the data from the occupational or leisure intercommunication comprises:
means for indicating an apparent health status improvement of the user at least partly based on a better-than-nominal performance in the occupational or leisure intercommunication.

80. The system of claim 55 in which the means for signaling a data distillation indicating a health status change of the user at least partly based on the data from the occupational or leisure intercommunication comprises:
means for obtaining the data distillation from the data from the occupational or leisure intercommunication and from other data.

81. The system of claim 55 in which the means for signaling a data distillation indicating a health status change of the user at least partly based on the data from the occupational or leisure intercommunication comprises:
means for responding to a selection of the data distillation.

82. A system comprising:
circuitry for obtaining data from occupational or leisure intercommunication at least between a device and a user; and
circuitry for signaling a data distillation indicating a health status change of the user at least partly based on the data from the occupational or leisure intercommunication.

83. The system of claim 82 in which the circuitry for obtaining data from occupational or leisure intercommunication at least between a device and a user comprises:
circuitry for receiving the data from the occupational or leisure intercommunication remotely.

84. (canceled)

85. The system of claim 82 in which the circuitry for obtaining data from occupational or leisure intercommunication at least between a device and a user comprises:
circuitry for receiving the data at the device.

86. (canceled)

87. The system of claim 82 in which the circuitry for obtaining data from occupational or leisure intercommunication at least between a device and a user comprises:
circuitry for receiving data from other interaction between the user and the device.

88. (canceled)

89. The system of claim 82 in which the circuitry for signaling a data distillation indicating a health status change of the user at least partly based on the data from the occupational or leisure intercommunication comprises:
circuitry for causing a module to process at least some user input from the data.

90-92. (canceled)

93. The system of claim 82 in which the circuitry for obtaining data from occupational or leisure intercommunication at least between a device and a user comprises:
circuitry for retrieving the data from a capture archive.

94. The system of claim 82 in which the circuitry for obtaining data from occupational or leisure intercommunication at least between a device and a user comprises:
circuitry for obtaining the data from one or more messages sent from the device.

95. The system of claim 82 in which the circuitry for obtaining data from occupational or leisure intercommunication at least between a device and a user comprises:
circuitry for receiving the data from one or more stationary sensors during the occupational or leisure intercommunication.

96. The system of claim 82 in which the circuitry for signaling a data distillation indicating a health status change of the user at least partly based on the data from the occupational or leisure intercommunication comprises:
circuitry for requesting an extraction of a portion of the data indicating when an ability of the user apparently changed.

97-98. (canceled)

99. The system of claim 82 in which the circuitry for signaling a data distillation indicating a health status change of the user at least partly based on the data from the occupational or leisure intercommunication comprises:
circuitry for distilling the data from the occupational or leisure intercommunication from other data.

100-102. (canceled)

103. The system of claim 82 in which the circuitry for obtaining data from occupational or leisure intercommunication at least between a device and a user comprises:
circuitry for obtaining optical information in the data from the occupational or leisure intercommunication.

104-105. (canceled)

106. The system of claim 82 in which the circuitry for signaling a data distillation indicating a health status change of the user at least partly based on the data from the occupational or leisure intercommunication comprises:
circuitry for indicating an apparent health status improvement of the user at least partly based on a better-than-nominal performance in the occupational or leisure intercommunication.

107-108. (canceled)
